(19) United States
(12) Patent Application Publication
(10) Pub. No.: US 2006/0199194 A1
(43) Pub. Date: Sep. 7, 2006

(54) Methods of detection using immuno-Q-Amp technology

(75) Inventors: Abraham Grossman, Pleasantville, NY (US); James Moe, New York, NY (US); Sara Vasan, New York, NY (US); Phyllus Y. Mong, Edgewater, NJ (US)

Correspondence Address: DAVIDSON, DAVIDSON & KAPPEL, LLC, 485 SEVENTH AVENUE, 14TH FLOOR, NEW YORK, NY, 10018, US

(73) Assignee: Q-RNA, Inc., New York, NY (US)

(21) Appl. No.: 11/237,057

(22) Filed: Sep. 28, 2005

Related U.S. Application Data
(63) Continuation-in-part of application No. 10/985,183, filed on Nov. 9, 2004.
(60) Provisional application No. 60/519,035, filed on Nov. 10, 2003.

(51) Int. Cl. C12Q 1/68; G01N 33/53

(52) U.S. Cl. 435/6; 435/7.1

(57) ABSTRACT

The present invention describes, in certain embodiments, a composition for detecting a tau protein comprising a modified detector molecule having two ends, a first end capable of binding the tau protein and a second end comprising a single-stranded DNA template, wherein the template is capable of being replicated by an RNA polymerase.

### RELATED APPLICATIONS

This application claims the benefit of and priority to U.S. provisional application No. 60/519,035, filed Nov. 10, 2003 and U.S. non-provisional application Ser. No. 10/985,183, filed Nov. 9, 2004, the disclosures of which are hereby incorporated by reference in their entireties.

### FIELD OF THE INVENTION

The present invention relates to methods of detecting target molecules using modified detector molecules wherein the modification facilitates signal amplification.

### BACKGROUND OF THE INVENTION

It is often desirable to detect the presence or absence of one or more relevant molecules in a complex sample. For example, it may be important to detect changes in a molecule that was subjected to certain conditions. Proteins can undergo conformational changes under certain conditions. These conformational changes can be important diagnostically providing an opportunity for early detection. Detecting such conformationally altered proteins can assist in the treatment of a particular disease or syndrome.

Some disease processes appear to stem from the misfolding of proteins. This misfolding is often associated with nucleic acids (NAs) and cellular factors. These misfolded proteins can go on to form pathological agglomerations. These agglomerations have been shown to be associated with neuronal cell death and brain wasting diseases such as Alzheimer's and Parkinson's disease in humans, scrapie, mad cow disease and chronic wasting diseases in animals. Spongiform encephalopathies, often involved with certain neuronal cell death and brain wasting syndromes, characteristically have protein plaques or agglomerations made manifest upon dissection. In spongiform encephalopathies, prion proteins are thought to be the etiologic agent. Prion-based diseases result from “infectious proteins” that are cellular benign prion proteins misfolded into an infectious isoform. This infectious isoform is involved in pathological protein agglomeration. Misfolded proteins also appear to damage cells in the lung, heart, kidney, pancreas and other organs.

The reliability of protein detection assays is limited by inadequate sensitivity and non-specificity. The target protein, which may be associated with a disease or condition, can be difficult to detect because it is often present in a biological sample in very low levels in a background of other biological molecules such as DNA, RNA and other non-target proteins. Furthermore, current protein detection assays are not readily adaptable to a form that is practical for clinical diagnostic use, because the assays are complicated and require special laboratory apparatus.

Typically, protein detection assays employ affinity ligands, which bind with high specificity to the target protein, and a reporter system that produces a detectable signal. The affinity ligand binds the target protein to form a complex. The formation of the complex is signaled with a reporter system which produces a detectable signal. Assays using antibodies as affinity ligands are called “immunoassays”. Popular examples of such immunoassays include: ELISA, immunohistochemistry and radioimmunoassay.

The sensitivity of a protein detection assay is dependent upon several factors; including the specificity of binding between the affinity ligand and the target protein in the assay, the specificity of the reporter system in producing a signal only from the complex formed in the assay, and the intensity of the signal generated by the reporter system in the assay.

With the advent of polymerase chain reaction (“PCR”), investigators developed an immuno assay that utilized a DNA reporter template that is amplified using PCR techniques (“immuno-PCR”). See, e.g., Sano, et al., 1992 Science 258:120-122 and U.S. Pat. No. 5,665,539. In immuno-PCR, the antibody binds the target protein followed by attachment of the template DNA molecule via a bi-specific linker molecule to form a protein-antibody-DNA conjugate.

Immuno-PCR technology includes techniques such as attaching template DNA molecules directly to the antibodies prior to initiating the assay (Joerger, et al., 1995 Clin. Chem.,41(9):1371-1377) and employing two monoclonal antibodies and a specific DNA template (Suzuki et al., 1995 Jpn. J. Cancer Res. 86:885-889). Typically, when using two antibodies, the first monoclonal antibody is immobilized, and the target protein is sandwiched between the first antibody and a biotinylated-second antibody. Free streptavidin is used to attach a biotinylated DNA to the second antibody. The biotinylated DNA complexed with antigen-antibody-streptavidin is amplified by PCR, and the products are analyzed by Southern blot analysis.

While these various immuno-PCR techniques provide high sensitivity, immuno-PCR generally, is still not readily adaptable for clinical diagnostic use. PCR, and hence immuno-PCR, procedures require numerous repeated steps, including: DNA strand separation, primer annealing, DNA polymerase-catalyzed replication, and extensive washings between these three steps. Furthermore, immuno-PCR requires cycling temperature regimes since each of these steps are sensitive to temperature. While immuno-PCR improved sensitivity, it has failed to become established as a routine method because of the extreme complexity of the technique, expensive reagents and disposables, and the requirement to have dedicated instrumentation, with prohibitively high installed cost.

Another immunoassay, immuno-RNA, uses template single-stranded RNA molecules for RNA-directed RNA polymerization to amplify the signal from the reporter system. Examples of the RNA polymerase include the replicase from bacteriophage Q-beta (Haruna and Spiegelman 1965 Science 150:884-886) and brome mosaic virus replicase (March et al., 1987 in: “Positive Strand RNA Viruses” Alan R. Liss, N.Y.). Q-beta replicase uses the RNA template to exponentially produce replicates RNA thereby producing a highly amplified reporter signal. A variety of chemical linkage methods for joining the template RNA to the affinity ligand have been employed.

However, immuno-RNA procedures have disadvantages. The template RNA molecules are notoriously unstable, being subject to degradation from RNases which are often present in the biological samples being tested, the laboratory equipment, and even dust in the air. Procedures involving RNA are typically performed under sterile conditions using scrupulously clean equipment and require chaotropic agents that inactivate RNases, such as guanidine isothiocyanate, guanidine hydrochloride, beta-mercaptoethanol and/or phenol/chloroform extractions. Procedures for synthesizing and handling RNA can be lengthy, hazardous and inconsistent.

Thus, an objective of this invention is to optimize the sensitivity and specificity of protein detection assays while also reducing the time and cost of these assays.

Certain embodiments of the present invention provide a novel means for detecting peptides that overcomes many of the disadvantages associated with previously known techniques. As is discussed in more detail, herein below, the presently described invention provides a more stable, easier to handle and control means for detecting peptides.

### BRIEF SUMMARY OF THE INVENTION

The present invention pertains to compositions and methods for detecting molecules from a complex sample using a modified detector molecule. In particular, the modified detector molecule is specific for a particular target molecule and comprises a DNA template that can be employed to amplify a signal indicating the presence of the target molecule. The use of the DNA template that is replicatable by a Q Beta replicase, provides advantages of sensitivity and specificity not previous achieved—allowing the present invention to detect nanogram levels of protein targets in a sample (equivalent to attomol concentrations). The present invention also provides a more stable means of detecting targets, and in particular proteins, that eliminates many of the contamination issues associated with other methods. Furthermore, the present invention is easier to control due to the ease with which the DNA template may be broken or inactivated.

In a preferred embodiment, the compositions and methods of the present invention comprise antibodies attached to template single-stranded DNA molecules which are replicable with a Q-beta replicase. It has been previously shown that Q-beta replicase has DNA-dependent RNA polymerase activity for both double-stranded and single-stranded DNA templates (Dimond, et al., U.S. Pat. No. 6,090,589). As mentioned above, the template ssDNA molecules are more stable and easier to handle compared to template RNA molecules. The methods disclosed herein are simple and do not require strand separation, primer annealing, cycling temperature regimes, or extensive washings. Furthermore, the Immuno Q-Amp compositions and methods produce billions of copies of replicated RNA molecules in less than 30 minutes, at room temperature, and require no special laboratory equipment. The present methods can be adapted for clinical diagnostics formats. Immuno Q-Amp also offers the added advantage of adaptability to in-the-field testing formats.

In one embodiment, the invention is directed to a composition for the detection of a target molecule comprising a modified detector molecule having two ends, a first end capable of binding to the target molecule, and a second detector end comprising a single-stranded DNA template, wherein the template is capable of being replicated by an RNA polymerase, also referred to herein as a replicase.

In another embodiment, the invention is directed to a composition for the detection of a target molecule comprising: (a) an affinity ligand capable of binding to the target; (b) a single-stranded DNA template capable of being replicated by an RNA polymerase; and

(c) a linker, wherein the linker links the affinity ligand to the template.

In yet another embodiment, the invention is directed to a method for detecting the presence of a target molecule, comprising: (a) contacting a sample to a modified detector molecule, such that a complex will be formed in the presence of the target, the modified detector molecule having two ends, a first end capable of binding to the target molecule, and a second detector end comprising a single-stranded DNA template, wherein the template is capable of being replicated by an RNA polymerase; (b) contacting the complex formed in (a) with an RNA replicase such that the DNA template will be replicated in the presence of the target; and (c) detecting the replicated DNA template thereby indicating the presence of the target molecule.

In a further embodiment, the invention is directed to a method for detecting the presence of a target molecule, comprising: (a) contacting a sample to an affinity ligand capable of binding the target; (b) further contacting the sample and the affinity ligand with a single-stranded DNA template capable of being replicated by a replicase, such that a complex will form in the presence of the target; (c) contacting the complex of (b) with a replicase such that the DNA template will be replicated in the presence of the target; and (d) detecting the replicated DNA template thereby indicating the presence of the target molecule.

In yet another embodiment of the invention, there is described a method of detecting a disease, comprising: (a) contacting a sample with a modified detector molecule, such that a complex will be formed in the presence of a target molecule that is indicative of the disease, the modified detector molecule having two ends, a first end capable of binding to the target molecule, and a second detector end comprising a single-stranded DNA template, wherein the template is capable of being replicated by a replicase; (b) contacting complex formed in (a) with a replicase such that the DNA template will be replicated in the presence of the target; and (c) detecting the replicated DNA template thereby indicating the presence of the target molecule and thus the presence of the disease.

One embodiment of the invention is directed to a method for detecting the presence or absence of a target molecule. The methods of the present invention comprise a detector molecule (or affinity ligand) having affinity for a target molecule. In one aspect, this detector molecule is modified. This modification can include the addition of a template molecule that serves as a template for replication facilitated by polymerase activity. In a further aspect of this embodiment, the template molecule is a polynucleotide. In one aspect, the polymerase is a DNA polymerase or a RNA polymerase. In a particular aspect, the polymerase is Q-Amp. In a particular aspect, the detector molecule is an antibody specific for a particular target molecule. In a still further aspect, the target molecule is a prion protein or fragment thereof.

Another embodiment is directed to a kit for performing the methods of the present invention. The kit comprises an affinity ligand specific for and having affinity to a target molecule. In one aspect, the affinity ligand has a template conjugated to it, wherein the template is a nucleic acid molecule. The kit further comprises a replicase activity. In one aspect, the replicase is Q-Amp.

For a further understanding of the present invention, together with other and further objects thereof, reference is made to the accompanying drawings and detailed description and its scope will be pointed out in the appended claims.

### BRIEF DESCRIPTION OF THE DRAWINGS

FIG. 1: depicts the basic concept of the present invention using isoforms of prion protein as an example;

FIG. 2: depicts the use of DNA templates in the present invention;

FIG. 3: depicts the use of RNA templates in the present invention;

FIG. 4: depicts one embodiment of the present invention.;

FIG. 5: depicts coating of a vesicle with antibody;

FIG. 6: depicts incubation with a detection antibody;

FIG. 7: depicts the results when Q-beta replicase is added;

FIG. 8: depicts an ELISA reaction;

FIG. 9: depicts the reaction product from an ELISA-Q-Amp protocol;

FIG. 10: depicts a “dipstick” kit;

FIG. 11: depicts a graph showing detection of nanogram quantities of human Tau target protein in fewer than 20 minutes at 37° C. using the Immuno Q-Amp method and testing a blocking reagent; and

FIG. 12: depicts a graph showing detection of nanogram quantities of human Tau target protein in fewer than 20 minutes at 37° C. using the Immuno Q-Amp method and testing a different blocking reagent. These results show high sensitivity and very low background signal.

### DETAILED DESCRIPTION OF THE INVENTION

The present invention pertains to methods of detecting molecules from a complex sample using a modified detector molecule. In particular, the modified detector molecule is specific for a particular target molecule and comprises a DNA template that can be employed to amplify a signal thus indicating the presence of the target molecule. These modified detector molecules comprise an affinity ligand joined with a deoxyribose nucleic acid template. The affinity ligand binds the target molecule. The nucleic acid template is replicable with a nucleic acid polymerase, thereby producing an amplified signal. Thus, the nucleic acid template serves as a reporter system when the affinity ligand binds the target molecule. The detector molecule described herein is also termed “Immuno Q-Amp”. The detector molecule is useful for detecting the presence or absence of the target molecule in a sample.

One embodiment of the invention is directed to a method for detecting the presence or absence of a target molecule. The methods of the present invention comprise a detector molecule having affinity for the target molecule. In one aspect, this detector molecule is modified. This modification includes the conjugation of a template molecule that can serve as a template for replication facilitated by RNA polymerase activity. In a further aspect of this embodiment, the template molecule is a polynucleotide. The template can be DNA, RNA, or modifications thereof. In one aspect, the polymerase is a DNA polymerase or a RNA polymerase. In a particular aspect, the polymerase is Q-Amp. In a particular aspect, the detector molecule is a modified antibody specific for a particular target molecule.

One embodiment of the invention is a composition for the detection of a target molecule comprising a modified detector molecule having two ends, a first end capable of binding to the target molecule, and a second detector end comprising a single-stranded DNA template, wherein the template is capable of being replicated by a RNA polymerase (or ‘RNA replicase’).

Another embodiment of the invention is a composition for the detection of a target molecule comprising: (a) an affinity ligand capable of binding to the target; (b) a single-stranded DNA template capable of being replicated by a RNA polymerase; and (c) a linker, wherein the linker links the affinity ligand to the template. (the linker would be to the detector or reporter antibody).

Another embodiment of the invention is a method for detecting the presence of a target molecule, comprising: (a) contacting a sample to a modified detector molecule, such that a complex will be formed in the presence of the target, the modified detector molecule having two ends, a first end capable of binding to the target molecule, and a second detector end comprising a single-stranded DNA template, wherein the template is capable of being replicated by a RNA polymerase; (b) contacting the complex formed in (a) with a replicase such that the DNA template will be replicated in the presence of the target; and (c) detecting the replicated DNA template thereby indicating the presence of the target molecule.

Another embodiment of the invention is a method for detecting the presence of a target molecule, comprising: (a) contacting a sample to an affinity ligand capable of binding the target; (b) further contacting the sample and the affinity ligand with a single-stranded DNA template capable of being replicated by a replicase, such that a complex will form in the presence of the target; (c) contacting the complex of (b) with a replicase such that the DNA template will be replicated in the presence of the target; and (d) detecting the replicated DNA template thereby indicating the presence of the target molecule.

A further embodiment of the invention is a method of detecting a disease, comprising: (a) contacting a sample with a modified detector molecule, such that a complex will be formed in the presence of a target molecule that is indicative of the disease, the modified detector molecule having two ends, a first end capable of binding to the target molecule, and a second detector end comprising a single-stranded DNA template, wherein the template is capable of being replicated by an RNA replicase; (b) contacting complex formed in (a) with an RNA replicase such that the DNA template will be replicated in the presence of the target; and (c) detecting the replicated DNA template thereby indicating the presence of the target molecule and thus the presence of the disease.

A further embodiment of the invention is a dipstick kit for detecting a target molecule comprising: (a) an affinity ligand capable of detecting the target molecule attached to a dipstick; and (b) a solution comprising a modified detector molecule capable of detecting the target molecule, attached to a liposome, wherein (i) the modified detector molecule comprises two ends, a first end capable of binding the target molecule and a second end comprising a single-stranded DNA template, wherein the template is capable of being replicated by an RNA polymerase and (ii) the liposome contains an RNA polymerase.

Another embodiment of the invention is a dipstick kit for detecting a target molecule comprising:(a) a modified detector molecule attached to a dipstick, wherein (i) the modified detector molecule comprises two ends, a first end capable of binding the target molecule and a second end comprising a single-stranded DNA template, wherein the template is capable of being replicated by an RNA polymerase; and (b) a solution comprising an affinity ligand capable of detecting the target molecule, attached to a liposome, wherein (i) the liposome contains an RNA polymerase.

In certain preferred embodiments, the single-stranded DNA is selected from the group consisting of MDV DNA, MNV DNA, MNV-AP1 DNA, MNVUP DNA, MNVLO DNA, RQ11+12 DNA and fragments and derivatives thereof.

In certain preferred embodiments, the single-stranded DNA is a positive strand or a negative strand.

In certain preferred embodiments, the RNA replicase (or polymerase) is a Q-beta replicase.

In certain preferred embodiments, the Q-beta replicase is a modified Q-beta replicase.

In certain preferred embodiments, the modified Q-beta replicase is Q-Amp.

In certain preferred embodiments, the single-stranded DNA is any single-stranded DNA capable of being replicated by Q-beta replicase.

In certain preferred embodiments, the first end of the modified detector molecule is an antibody.

In certain preferred embodiments, the antibody is a monoclonal antibody.

Embodiments of the invention include a target that is a protein, a nucleic acid, or a lipid.

In certain preferred embodiments, the target is a protein.

In certain preferred embodiments, the protein is indicative of a disease.

In certain preferred embodiments, the protein is a prion protein.

In certain embodiments, the protein is tau.

In certain embodiments, the protein is a misfolded tau protein.

In certain embodiments, the presence of a misfolded tau protein is indicative of a neurogenic disorder, such as Alzheimer's disease.

In certain preferred embodiments, the prion protein is a scrapie prion protein.

In certain preferred embodiments, the template is further attached to a detectable label.

In certain preferred embodiments, the label is a fluorescent label.

In certain preferred embodiments, the label is a radioactive label.

In certain preferred embodiments, the linker is a covalent bond.

In certain preferred embodiments, the linker comprises biotin and Avidin or its derivatives.

In certain preferred embodiments, the methods and kits described herein can be used at ambient temperature.

In certain preferred embodiments, the methods and kits described herein can be used under thermo-cycling conditions.

The present invention provides methods for detecting in a sample (e.g., a biological sample) the presence or absence of a target molecule, such as a protein, nucleic acid, lipid, carbohydrate, or a fragment or derivative thereof or any combination thereof.

In one embodiment, the method comprises: contacting the modified detector molecule with the sample under conditions that are suitable for the modified detector molecule to bind with the target protein to form a complex comprising the antibody portion of the modified detector molecule bound to the protein; and reacting the complexed protein detector molecule with Q-beta replicase or with Q-Amp replicase under conditions that are suitable for the replicase to produce replicated nucleic acid molecules.

In another embodiment, the method comprises: contacting the sample with a capture antibody under conditions that are suitable for the capture antibody to bind with the target protein; contacting the protein detector molecule with the captured target protein under conditions that are suitable for the protein detector molecule to bind with the captured target protein to form a complex comprising the capture antibody/target protein/detector molecule; and reacting the complexed protein detector molecule with Q-beta replicase or with Q-Amp replicase under conditions that are suitable for the replicase to produce replicated nucleic acid molecules. The capture antibody can be bound to a solid support.

In all these embodiments, the presence of the target protein within the sample is indicated by the production of replicated nucleic acid molecules. The replicated nucleic acid molecules, which are produced by the replicase, can be labeled with a detectable marker to “report” the presence of the target protein.

The replicase can produce the replicated nucleic acid molecules in the presence of NTPs and/or rNTPs which can be labeled with a detectable marker including a radioisotope, a fluorescent compound, a bioluminescent compound, a chemiluminescent compound, a metal chelator or an enzyme. Technologies for generating labeled nucleic acid molecules are well known (Sambrook et al., 1989 in “Molecular Cloning”).

The present “Immuno Q-Amp” invention provides compositions and methods that are advantageous over standard ELISA-based immunoassays. The Immuno Q-Amp invention herein disclosed is highly sensitive, being capable of detecting nanogram levels of protein targets in a sample (equivalent to attomol concentrations), because the replicase enzyme generates billions of replicated nucleic acid strands in fewer than 30 minutes. Additionally, it has been previously demonstrated that methods using Q-beta replicase enzyme are useful for linear quantification having a range of sensitivity up to 10⁹-fold of target protein concentration (Lizardi, et al., 1988 BioTechnology 6:1197-1202).

The Immuno Q-Amp methods are also simple compared to immuno-PCR methods because the replicase enzyme does not require primers and catalyzes RNA replication at a range of temperatures, including room temperature to 37° C., at normal pH. Thus, the Immuno Q-Amp methods do not require cycling temperature regimes, primer annealing, strand separation, or extensive washing steps.

Thermo-Cycling

While one of the advantages of the present invention is that it is not dependent upon temperature, and in fact can be used at room temperature, in certain embodiments of the invention thermocycling can be used to either reduce background noise or otherwise control the procedure to obtain more desirable results in ways that would be appreciated by one of skill in the art. Thus, embodiments of the present invention include using the invention under isothermal or thermocycling conditions, or a combination of both. Under the isothermal or thermo-cycling conditions, the methods can be performed at any temperature(s), but most preferably in temperatures ranging of from about 20°-60° C. One skilled in the art can select the thermo-cycling conditions suitable for the particular template nucleic acid molecule used, or for selecting a desired replication product.

The Immuno Q-Amp methods also provide consistent results compared to immuno-RNA methods, because the Immuno Q-Amp methods use template DNA which is more stable and easier to handle than RNA. The Immuno Q-Amp methods do not require scrupulously clean equipment and chaotropic agents that inactive RNases.

Unlike existing protein detection methods, the sensitivity, simplicity and consistency of the Immuno Q-Amp methods make it readily adaptable to a clinical diagnostic format.

As used herein, the term “affinity” means exhibiting an attraction or capacity for binding. A specific affinity is an attraction that is directed to a particular feature or sequence of a molecule.

As used herein, the terms “polynucleotide” and “oligonucleotide” are used interchangeably, and include polymeric forms of nucleotides of any length, either deoxyribonucleotides or ribonucleotides, or analogs thereof. Polynucleotides can have any three-dimensional structure, and can perform any function, known or unknown. The following are non-limiting examples of polynucleotides: a gene or gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide can comprise modified nucleotides, such as methylated nucleotides and nucleotide analogs. If present, modifications to the nucleotide structure can be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be inputted into databases in a computer having a central processing unit and used for bioinformatics applications such as functional genomics and homology searching.

The term “protein” includes a compound of two or more subunit amino acids, amino acid analogs, peptides, or peptidomimetics. The subunits may be linked by peptide bonds. In another embodiment, the subunit may be linked by other bonds, e.g., ester, ether, etc. As used herein the term “amino acid” includes either natural and/or unnatural or synthetic amino acids, including glycine and both the D or L optical isomers, and amino acid analogs and peptidomimetics. A peptide of three or more amino acids is commonly referred to as an oligopeptide. Peptide chains of greater than three or more amino acids are referred to as a polypeptide or a protein.

As used herein, “fragment” refers to a molecule that originates from a parent polymeric molecule. A fragment is a processed molecule that can serve partially of completely as the parent polymeric molecule. It shares many of the features that are characteristic of the parent.

As used herein, “derivative” refers to a molecule that has been modified in some physiochemical fashion as compared to the parent molecule. For example, chemical groups can be added (or eliminated) to the parent molecule thus rendering a derivative of the parent.

The target molecule of the present invention includes proteins (peptides, etc.), nucleic acids, lipids, any combinations thereof, as well as any fragments or derivatives thereof. In a preferred embodiment of the invention the target is a protein that is indicative of a disease state. The target molecule needs to have sufficient complexity to allow it to interact with components of an immune system, such as an antibody. In one aspect of the present invention, the target molecule is a prion protein. In a particular aspect, the prion protein (“PrP”) is the pathological scrapies form of the protein (“PrP-SC”). In another embodiment, the target protein is a tau protein (see review by: Johnson and Stoothoff 2004 J. of Cell Sci 117:5721-5729). However, one of skill in the art will appreciate the applicability of the present invention to any target.

Tau proteins form a group of isoforms produced by alternative RNA splicing (Johnson and Jenkins 1999 J. Alzheimer's Dis 1:307-328; Kosik et al., 1989 Neuron 2:1389-1397). Tau proteins are predominantly neuronal proteins. In adult human brain, there are six major isoforms of tau. In general, tau proteins have an acidic N-terminal domain, a proline-rich domain, a microtubule-binding repeat domain, and a C-terminal domain. Tau proteins are neuronal, microtubule-associated, phospho-protein that play key roles in regulating microtubule dynamics (Weingarten et al., 1975 Proc Natl Acad Sci USA 72:1858-1862; Watanabe et al., 1993 J. Biol. Chem. 268:25712-25717), axonal transport and neurite outgrowth. These normal functions of tau are modulated by site-specific phosphorylation.

Evidence indicates that hyper-phosphorylation of tau causes tau oligomerization and accumulation of filamentous aggregates of tau (Grundke-Iqbal et al., 1986 J. Biol. Chem. 261:6084-6089; Grundke-Iqbal et al., 1986 Proc Natl Acad Sci USA 83:4913-4917; Kosik et al., 1986 Proc Natl Acad Sci USA 83:4044-4048; Wood et al., 1986 Proc Natl Acad Sci USA 83:4040-4043). Abnormal tau phosphorylation reduces tau binding to microtubules (Biernat et al., 1993 Neuron 11:153-163; Biemat and Mandelkow, 1999 Mol. Biol. Cell 10:727-740; Drewes et al., 1995 J. Biol. Chem. 270:7679-7688; Cho and Johnson, 2003 J. Biol. Chem. 278:187-193), interferes with microtubule assembly (Cleveland et al., 1977 J. Molec. Biol. 116:227-247; Jameson et al., 1980 Biochemistry 19:2472-2479; Lindwall and Cole, 1984 J. Biol. Chem. 259:5301-5305; Iqbal et al., 1986 Lancet 2:421-426), neurite outgrowth, and axonal transport. The tau aggregates are believed to be a contributing factor to neurodegenerative diseases, such as Alzheimer's disease.

In one embodiment, the tau protein is any isoform of tau. The tau protein can be from any age subject including fetal, infant, child, adolescent or adult. The tau protein can be a normally phosphorylated or abnormally phosphorylated protein.

The target molecule can be a protein which undergoes misfolding. The misfolded protein can form amyloid or fibril deposits in any part of the subject, such as the brain, lung, heart, kidney, pancreas or other organs. The misfolded protein can be associated with a benign condition or a disease in a subject such as neurodegenerative diseases (Alzheimer's, Parkinson's, amyotrophic lateral sclerosis, Huntington's, Pick's), metabolic diseases (diabetes), ocular diseases (cataract), or pulmonary diseases (cystic fibrosis). Other neurodegenerative diseases include transmissible spongiform encephalopathies (TSEs) which affect the central nervous system (CNS). TSEs include: scrapies in sheep; Bovine Spongiform Encephalopathy (BSE) in cattle; and Creutzfeldt-Jakob Disease (CJD), Guerstmann-Straussler-Scheinker Syndrome (GSS), kuru, and Fatal Familial Insomnia (FFI) disease in humans.

Common to all of the TSE diseases is long incubation periods and the accumulation of amyloid-like rods or scrapie associated fibrils (SAPs). The formation of SAPs is the result of extensive fibrillation of PrP^{sc}, the isoform of the endogenous and innocuous PrP^{c} protein (cellular protein—physiologically normal) that is associated with infectivity. The structural transformation of the soluble PrP^{c} to the insoluble PrP^{sc} isoform marks the onset and progression to clinical prion disease.

In one embodiment, the target molecule is a prion protein (e.g., PrP). The PrP protein can be in any state including cellular (PrP^{C}), scrapies (PrP^{SC}), or resistant (PrP^{RES}). The PrP protein can be found as part of a heterologous population having any combination of these PrP states. The PrP protein can be proteinase-K resistant. The PrP protein can be folded into a predominantly alpha-helix or beta sheet conformation. The PrP protein can be in a soluble or insoluble state. The PrP protein can be a misfolded PrP protein.

The gene that encodes PrP^{c} is highly conserved and constitutively expressed from the prnP locus as a 35 kDA glycoprotein (Chesebro et al., 1985 Nature 315:331-33; Oesch et al., 1985 Cell 40:735-46; the entire teachings of which are incorporated herein by reference). Approximately one half of translated prPC is processed to the extracellular membrane where it is anchored to the plasma membrane by a C-terminal glycosyl-phophatidyl-inositol (GPI) anchor. However, PrP^{C} has also be found in two trans-membrane forms, one with the N-terminus inside the ER lumen (PrP^{Ntm}; 40-50%) and the other in the opposite orientation with the C-terminus inside the ER lumen (PrP^{Ctm}; 10%). It is unknown if these processing differences reflect the functional properties of these PrP^{c} forms.

PrP proteins have interesting structural characteristics, particularly the extraordinary transformation from its native, wildtype conformation PrP^{c} to the infectious PrP^{sc}. The alteration in protein structure is marked by a transition from alpha-helix rich of PrP^{c} into beta-sheet rich regions in the C-terminal domain of the isoform associated with infectivity (Pan et al., 1993 PNAS, USA 90:10962-66; the entire teachings of which are incorporated herein by reference). Several biochemical traits distinguish the isoforms such as the insolubility of PrP^{sc} in physiologic solutions and the resistance of its C-terminal domains (amino acids 90-231) to digestion by proteinase K. The structure of the non-protease treated full-length N-terminus of PrP is very flexible and without a single, stable structure based on NMR structural studies. Therefore, this region of PrP is most likely indistinguishable between the cellular and scrapie isoforms.

Binding of PrP to nucleic acids has been demonstrated many times through the observation of direct complex formation in vitro with purified protein and by copurification of nucleic acids from scrapie associated fibrils (SAFs) removed from infected tissue (Merz et al., 1981 Acta Neuropathol (Berl) 54(1):63-74; the entire teachings of which are incorporated herein by reference). It is this binding that may be responsible for converting the cellular form to the pathological scrapies form. For example, several thousand bases of the viral RNA genome of IAP were co-purified with SAF from infected tissue (Murdoch, et al., 1990 Virology 64(4): 1477-86; Akowitz et al., 1994 NAR 22(6): 1101-07; the entire teachings of which are incorporated herein by reference). Such observations influenced studies to explore the possibility that nucleic acids were a required genetic component in the transmission of TSE, although no such genetic link has been experimentally determined to date.

Indirect evidence for an in vivo association between PrP and viral components is the observation that the rate of PrP^{sc} formation is accelerated in cells affected with moloney murine leukemia virus (Carp et al., 1999 J Gen Virol 80(pt 1):5-10; the entire teachings of which are incorporated herein by reference). There is further evidence of interactions between PrP and viral nucleic acids derived from in vitro studies that used recombinant, mammalian PrP proteins expressed in E. coli. Syrian Golden Hamster recombinant PrP^{c} (srPrP) has a surprising homology of in vitro activities with the nucleocapsid protein from HIV (Ncp7) (Tanchou et al., 1995 J Mol Biol 252:563-71; Gabus et al., 2001a J Mol Biol 307(4):1011-21; Gabus et., 2001b J Biol Chem 276(22): 19301-9; the entire teachings of which are incorporated herein by reference). srPrP has virtually the same level of activity as Ncp7 in the processes of DNA strand-transfer, nucleic acid chaperoning, HIV-RT priming, and the formation of condensed protein/nucleic acid structures. Double stranded DNA also induced the formation of similar condensed PrP structures, as well as resistance to proteinase K digestion (Nandi, 1998 Arch Virol 143(7):1251-63; Nandi and Leclerc, 1999 Arch Virol 144(9):1751-63; Nandi and Sizaret, 2001 Arch Virol 146:327-45; the entire teachings of which are incorporated herein by reference). Recently, two small RNA aptamers have been isolated based on their ability to bind to PrP proteins. One aptamer, API (29 nt), was isolated using recombinant srPrP, and is predicted to fold into a compact structure containing three stacked G-quartets, a structure suggested to be important in binding to srPrP (Weiss et al., 1997 J Virol 71(11):8790-97; the entire teachings of which are incorporated herein by reference). Using a series of srPrP truncations, the authors localized the binding of API to the flexible N-terminus, within amino acids 23-39.

Biological Samples

The present invention provides detector molecules that detect the presence or absence of a target molecule in a sample. In one embodiment, the sample is a biological sample from a subject. The biological sample can be from any body part of the subject including brain, lymph nodes, spleen, tonsil, peripheral nerve, or spine. The biological sample can be any tissue or biological fluids from a subject including, but is not limited to, tissue extracts, urine, blood (plasma, serum, or platelets), phlegm, saliva, or cerebrospinal fluid.

The methods of the present invention can be used to detect the presence or absence of a target molecule using modified detector molecules. In one aspect, the modified detector molecule is an antibody. The antibody population can be either monoclonal antibodies, polyclonal antibodies, or a combination thereof. In one aspect, the antibody has affinity with PrP^{sc} or tau.

Affinity Ligands

The present invention provides detector molecules comprising an affinity ligand, such as an antibody, attached to a nucleic acid which is replicable with a nucleic acid polymerase. Other examples of affinity ligands include antigens for binding antibody targets, and lectins for binding glycoproteins or polysaccharides.

The antibody can be polyclonal, monoclonal, chimeric, humanized, human, an anti-idiotypic antibody, an immunologically-active fragment or derivative thereof, a recombinant protein having immunologically-activity, or a labeled antibody.

The antibody can bind selectively to any of the target proteins embodied above. Preferably, the antibody will not bind, or will bind weakly, to a non-target protein. The present invention provides detector molecules comprising antibodies which can be from any source, including rabbit, sheep, goat, rat, mouse, dog, cat, pig, horse, monkey, ape and human.

Polyclonal Antibodies

The polyclonal antibodies include a population of different antibodies directed against a different epitope on the target protein. Polyclonal antibodies can be produced by methods well-known in the art. Polyclonal antibodies can be produced by immunizing animals, usually a mammal, by multiple injections of an immunogen and an adjuvant (Harlow and Lane, 1988, in: “Antibodies: A Laboratory Manual” Cold Spring Harbor Press). In general, any antibody (e.g., monoclonal, polyclonal, and the like) can be raised using an isolated target protein, or a fragment as the immunogen. Cells expressing or overexpressing the target protein can also be used for immunizations. Similarly, any cell engineered to express a target protein can be used. Administration of the immunogen is conducted generally by injection into an animal over a suitable time period and with use of a suitable adjuvant, as is generally understood in the art (B S Dunbar and E D Schwoebel 1990 Methods Enzymol 182:663-670).

The full-length or fragments of the target proteins can be used as an immunogen to produce the polyclonal antibodies. Alternatively, the amino acid sequence of any of the target protein can be used to select specific regions of these polypeptides for generating antibodies. For example, hydrophobicity and hydrophilicity analyses of these amino acid sequences can be used to identify hydrophilic regions. These amino acid sequences that show immunogenic structure, as well as other regions and domains, can readily be identified using various other methods known in the art (B Rost and C Sander 1994 Protein 19:55-72), such as Chou-Fasman, Garnier-Robson, Kyte-Doolittle, Eisenberg, Karplus-Schultz or Jameson-Wolf analysis. Methods for preparing an immunogen and for preparing immunogenic conjugates of a protein with a carrier such as BSA, KLH, or other carrier proteins are well known in the art.

The animals are typically immunized with about 1 micro gram to about 1 milligram of immunogen capable of eliciting an immune response, along with an enhancing carrier preparation, such as Freund's complete adjuvant, or an aggregating agent such as alum to produce an immunogen mixture. The immunogen mixture can be injected into the animal at multiple sites. Additional booster injections can be administered to the animal with at least one subsequent administration of a lower amount of the immunogen mixture which include about ⅕ to 1/10 the original amount of the immunogen in Freund's complete adjuvant (or other suitable adjuvant). Typically, the animals are bled, the serum is assayed to determine the specific antibody titer, and the animals can be boosted again and assayed until the titer of antibody no longer increases.

The polyclonal antibody serum can be collected using well known methods or the antibody fraction can be enriched by chromatography with an affinity matrix that selectively binds immunoglobulin molecules, such as protein A, to obtain the IgG fraction. The enriched polyclonal antibody can be further enriched using immunoaffinity chromatography such as solid phase-affixed immunogen. For example, the enriched polyclonal antibody fraction is contacted with the solid phase-affixed immunogen for a period of time sufficient for the immunogen to immunoreact with the antibody molecules to form a solid phase-affixed immunocomplex. The bound antibodies are eluted from the solid phase by standard techniques, using of buffers of decreasing pH or increasing ionic strength. The eluted fractions are assayed, and those including the specific antibodies are combined.

Monoclonal Antibodies

Monoclonal antibodies are typically produced as homogenous populations and bind with a single determinant on an antigen. Methods for producing monoclonal antibodies are well known in the art and can be produced using any of several different technologies. For example, monoclonal antibodies can be produced using hybridoma technology (Kohler and Milstein 1975 Nature 256:495-497; Brown et al. 1981 J Immunol 127:539-46; Brown et al., 1980 J Biol Chem 255:4980-83; Yeh et al., 1976 Proc Natl Acad Sci USA 76:2927-31; Yeh et al., 1982 Int J Cancer 29:269-75), or human B cell hybridoma techniques (Kozbor et al., 1983 Immunol Today 4:72), or EBV-hybridoma techniques (Cole et al., 1985 Monoclonal Antibodies and Cancer Therapy, Alan R. Liss, Inc., pp. 77-96), or recombinant DNA methods in bacteria, animal cells or plant cells (U.S. Pat. No. 4,816,567), or phage antibody libraries (Clackson, et al., 1991 Nature 352:624-628; Marks, et al., 1991 J Mol Biol 222:581-597). The monoclonal antibodies can be made using a repetitive, multiple site immunization strategy termed RIMMS (K E Kilpatrick, et al., 1997 Hybridoma 16:381-389). An alternative method includes producing affinity matured monoclonal antibodies by fusing a myeloma cell line stably transfected with Bcl-2 and immune lymphocytes (K E Kilpatrick, et al., 1997 Hybridoma 16:381-389).

The hybridoma cell secreting the desired antibodies can be screened by immunoassay in which the antigen is the target polypeptide. When the appropriate hybridoma cells secreting the desired antibody are identified, the cells can be cultured either in vitro or by production in ascites fluid. The desired monoclonal antibodies are then recovered from the culture supernatant or from the ascites supernatant.

Chimeric Antibodies

The chimeric antibodies comprise an antibody portion (e.g., immunoglobulin portion) from one species or a particular antibody class or subclass, joined to an antibody portion from a different species or antibody class or subclass. The chimeric antibodies can be produced as CDR grafted antibodies of multiple species origin. The portions of the chimeric antibodies can be from any source, including bovine, porcine, murine, equine, canine, feline, monkey, ape, piscine, ovine, avian or human. In particular, the portions of the chimeric antibodies can be from a mammal including rabbit, sheep, goat, rat, mouse, dog, cat, pig, horse, monkey, ape or human.

For example, one portion of the chimeric antibody can include a constant immunoglobulin portion from one species, and another portion includes a variable region (e.g., antigen combining region). The chimeric antibody comprises hypervariable loop regions from one species and invariant framework regions from another species. Chimeric antibodies comprising human regions are useful, as they are less likely to be antigenic to a human subject than antibodies with non-human constant regions and variable regions. The chimeric antibodies can be produced by methods known in the art (Morrison et al., 1985 Proc Natl Acad Sci USA 81:6851; Takeda et al., 1985 Nature 314:452; Cabilly et al., U.S. Pat. No. 4,816,567; Boss et al., U.S. Pat. No. 4,816,397; Schenk et al., U.S. Pat. No. 6,750,324).

The chimeric antibodies can be chimeric proteins having distinct antigen binding specificities (Boulianne et al., 1984 Nature 312:643; Sahagan et al., 1986 J Immunol 137:1066). The chimeric antibodies can be chimeric proteins having different effector functions (Neuberger et al., 1984 Nature 312:604), or immunoglobulin constant regions from another species and constant regions of another immunoglobulin chain (Sharon et al., 1984 Nature 309:364; Tan et al., 1985 J Immunol 135:3565-3567). Chimeric antibodies can be produced using homologous recombination to target gene modification (Fell et al., 1989 Proc Natl Acad Sci USA 86:8507-8511).

Humanized Antibodies

The humanized antibodies comprise antibody portions from a human immunoglobulin. In one embodiment, a humanized antibody comprises hypervariable loop regions and/or invariant framework regions from human. In one embodiment, a humanized antibody comprises hypervariable loop regions from non-human species and invariant framework regions from human. A humanized antibody can comprise at least a portion of an immunoglobulin constant region from human. Humanized antibodies can be made according to any known method, including substituting one or more of the non-human antibody CDRs for corresponding human antibody sequences (Teng et al., 1983 Proc Natl Acad Sci USA 80:7308-7312; Kozbor et al., 1983 Immunology Today 4:7279; Olsson et al., 1982 Meth Enzymol 92:3-16; Jones 1986 Nature 321-522-525; Riechmann, et al., 1988 Nature 332:323-329; Verhoeyen et al., 1988 Science 239: 1534-1536; Presta 1992 Curr Op Struct Biol 2:593-596; Carter et al., 1993 Proc Natl Acad Sci USA 89: 4285; Sims et al., 1993 J Immunol 151: 2296).

Humanized antibodies can be produced using transgenic animals such as mice which are incapable of expressing endogenous immunoglobulin heavy and light chain genes, but which can express human heavy and light chain genes. The transgenic mice are immunized with a selected antigen, such as the target protein (or a fragment or derivative thereof). The human immunoglobulin transgenes harbored by the transgenic mice rearrange during B cell differentiation, and subsequently undergo class switching and somatic mutations. This technology produces IgG, IgA, and IgB antibodies (Lonberg and Haszar (1995 Int Rev Immunol 13:65-93; and U.S. Pat. Nos. 5,625,126; 5,633,425; 5,569,825; 5,661,016; and 5,545,806.

The humanized antibody can be more fully-humanized or fully humanized. These antibodies can be produced using methods known in the art (Vaughan et al., 1998 Nature Biotechnology 16: 535-539; Griffiths and Hoogenboom, “Building an in vitro immune system: human antibodies from phage display libraries”, in: Protein Engineering of Antibody Molecules for Prophylactic and Therapeutic Applications in Man. Clark, M. (Ed.), Nottingham Academic, pp 45-64 (1993); Burton and Barbas, Human Antibodies from Combinatorial Libraries Id., pp 65-82).

Recombinant Protein

The modified detector molecules can include recombinant proteins which exhibit the functional activity of an antibody (e.g., bind a target protein, or fragments or derivatives thereof). The recombinant proteins can be produced by a cell engineered to express the recombinant protein. The recombinant protein can be produced by methods used to produce conventional antibodies, such as polyclonal technology, hybridoma technology, and/or phage library technologies (R D Mayforth and J Quintans 1990 New Eng J Med 323:173-178; TA Waldmann 1991 Science 252:1657-1662; G Winter and C Milstein 1991 Nature 349:293-299; S L Morrison 1992 Ann Rev Immunol 10:239-266).

The recombinant proteins can be a single chain polypeptide molecule that bind the target protein. The heavy (H) and light (L) chains of an Fv portion of an antibody can be encoded by a single nucleotide sequence and include a linker sequence (Bird et al. 1988 Science 242:423-426; Huston et al. 1988 Proc Natl Acad Sci USA 85:5879-5883).

The recombinant proteins can be mono-specific or bispecific. The bi-specific proteins will have one portion that binds the target polypeptide and another portion will bind a different target polypeptide. The mono-specific proteins have one portion that binds the target polypeptide.

Antibodies That Competitively Inhibit

The antibodies can be any antibody which competitively inhibits the immunospecific binding of any of the above-described antibodies to the target polypeptide. The competitive inhibiting antibody can bind to the same epitope as the epitope bound by the above-described antibodies. These competitive inhibiting antibodies can be identified by routine competition assays (Harlow, E. and Lane, D. 1988 Antibodies, A Laboratory Manual, Cold Spring Harbor Laboratory, Cold Spring Harbor, N.Y.). For example, the competition assays can be a competitive ELISA assay. It is appreciated by those in the art that other competition assays can be performed.

Anti-Idiotypic Antibodies

The antibodies can be anti-idiotypic antibodies that mimic the target protein. The anti-idiotypic antibodies bind an idiotype on any of the above-described antibodies. Methods for producing anti-idiotypic antibodies are well known in the art (Wagner et al., 1997 Hybridoma 16: 33-40; Foon et al., 1995 J Clin Invest 96: 334-342; Herlyn et al., 1996, Cancer Immunol Immunother 43: 65-76).

Antibody Fragments

The antibody can be an antibody fragment that recognizes and binds the target protein. An antibody fragment comprises a portion of an intact antibody, such as, for example, the antigen-binding or variable region of the intact antibody. The antibody fragment can comprise the constant region of the intact antibody. Antibody fragments include Fab, Fab′, F(ab′)₂, or Fv fragments (U.S. Pat. No. 5,641,870; Zapata, et al. 1995 Protein Eng 8:1057-1062). Antibody fragments also include single-chain antibodies and recombinant proteins which bind the target protein. The antibody fragments can be generated by papain digestion of intact antibodies to produce Fab and Fc fragments, or by pepsin digestion to produce F(ab′)₂ fragments.

A monoclonal antibody (mAb) according to the present invention is intended to bind to, detect and qualitatively and quantitatively measure the presence of epitopes of prion proteins whether they are in soluble or insoluble form in various tissue specimens such as homogeneous or sections of brain, spleen, tonsils, white blood cells others and body fluids such as blood, cerebrospinal fluid saliva, urine or others. The present mABs bind to epitopes of amino acids in a row or to epitopes of amino acids on different loops of the three-dimensional structure of native PrPs which are spatially close to each other. A particular group of the present antibodies binds only to native disease-specific PrP and not to native normal PrP.

The term monoclonal antibody comprises also chimeric monoclonal antibodies having similar properties, which are derived from different animals, such as human/mouse chimeric antibodies or any other chimeric molecule comprising the antigen-binding part of the monoclonal antibody (idiotype) with other molecules such as antibody fragments of other monoclonal antibodies or enzymes. See, U.S. Pat. No. 6,750,324, the entire teaching of which is incorporated herein by reference.

A fragment of a monoclonal antibody comprising the binding part of the monoclonal antibody (idiotype) likewise capable of specifically binding the antigen and is termed Fab or (Fab′)₂ depending on whether the monoclonal antibody is digested with papain or pepsin, respectively.

A synthetic antibody or fragments thereof designed according to the amino acids or substituted homologous amino acids composing the idiotype responsible for binding the antigen. Homologous amino acids are defined as exchanges within the following five groups. (1) Small aliphatic, nonpolar or slightly poor residues: alanine, serine, threonine, glycine, proline; (2) Polar, negatively charged residues and their amides: aspartic acid, asparagine, glutamic acid, glutamine; (3) Polar, positively charged residues: histidine, arginine, lysine; (4) Large aliphatic, nonpolar residues: methionine, leucine, isoleucine, valine, cysteine; (5) Large aromatic residues: phenylalanine, tyrosine, tryptophan. In one aspect the monoclonal antibodies of the present invention are 6H4, 34C9, 15B3 which are produced by hybridoma cell lines DSM ACC2295, DSM ACC2296 and DSM ACC2998, respectively. See, U.S. Pat. No. 6,765,088, the entire teaching of which is incorporated herein by reference.

In certain embodiments, the antibodies and fragments thereof are essential tools for immunological detection procedures based on the binding of the prion protein to the presented monoclonal antibodies in an antigen-antibody complex. The monoclonal antibodies, in certain embodiments of the present invention, react with recombinant PrP as well as native or denatured Prp^{c} and Prp^{sc} whether they are in soluble or insoluble state. The monoclonal antibodies react further on with PrP from different species, for example, humans, hamsters, pigs, sheep, cattle and mice. Furthermore, the present antibodies by forming an antigen-antibody complex between the presented monoclonal antibodies and the prion protein can be used to inhibit neurotoxic and infectious properties of the disease-specific prion protein.

The current invention relates to anti-idiotype antibodies which are antibodies that bind with their binding region (idiotype) to the binding region of the original monoclonal antibody. The anti-idiotype antibody mimics features of the original antigen, in this case features of PrP. Anti-idiotype antibodies are raised as polyclonal antibodies (serum) or monoclonal antibodies from animals immunized with the antibodies according to the invention. Anti-idiotype antibodies are valuable tools in detecting and blocking interactions of, for example, the original antigen (PrP), particularly interactions with receptors and can therefore be used in prevention and therapy of, in this example, prion diseases.

A stable hybridoma cell line according to the present invention is capable of producing a monoclonal antibody as defined above over a prolonged time period of at least six months. Such cell lines are derived from the fusion of a spleen cell expressing the antibody derived from mice lacking a functional PrP gene, and a myeloma cell of mice providing survival of the fused cell lines using methods well known to those skilled in the art.

Suitable examples of hybridoma cell lines are DSM ATC2295, DSM ACC2296 and DSM ACC2298. The first two cell lines were deposited under the Budapest Treaty on Feb. 6, 1997 at the Deutsch Sammlung von Mikroorganisnien und Zellkulturen GmbH, Mascheroder Weg 1b, D-38124 Braunschweig, a recognized public depository for strains of microorganisms. The hybridome cell line producing mAB 15B3 was deposited Feb. 13, 1997 under number DSM ACCC2298 at the same depository.

The present method for the production of an antibody according to the invention comprises culturing a hybridoma cell line as mentioned above and isolating the monoclonal antibody from the supernatant of the growth media.

Culturing is carried out in a flasks in HT-medium or in a cell culturing system called “technomouse” in serum-free, synthetic medium (Turbodoma medium, supplied by Messi, Zurich). In a “technomouse” hybridoma cells are cultured in a sterile chamber surrounded by a protein-impermeable membrane that is perfused by the respective medium in a constant flow rate (for example, turbomedium at 80 ml/h); antibodies are collected from the chamber with the help of a syringe at regular intervals. Isolation of monoclonal antibodies is carried out by extraction from the supernatant by conventional biochemical methods, e.g., by use of affinity columns with the corresponding immobilized antigen or by any other method used in the art, such as gel filtration or ion exchange chromatography. In the “technomouse” supplied with serum-free turbomedium antibody concentrations and purities are achieved that need no further extracting procedures.

Chimeric antibodies and fragments thereof can be produced by genetic engineering methods, e.g., by sequencing the antibody or the desired fragment thereof and constructing DNAs coding for the chimeric antibody or the fragment thereof which DNAs are inserted into an appropriate expression vector and expressed to produce the antibody or the fragment thereof in both procaryotic or eukaryotic cell lines.

For example, a fragment binding to a PrP epitope can be combined with a human heavy chain to produce chimeric antibodies for use in humans as therapeutic or preventive agents against a prion disease. A fragment binding to a PrP epitope can also be combined with other enzymes, proteins or molecules to give rise to chimeric molecules combining the biological functions of these, e.g., for targeting an enzymatic activity to a place defined by the proximity of the PrP epitope.

In an embodiment, the present method for the production of a hybridoma cell line comprises administering to PrP^{0/0} mice (knockout mice without a functional PrP gene) an immunizing amount of a recombinant pure prion protein PrP, removing the spleen from the immunized mice, recovering splenocytes therefrom, fusing the latter with appropriate myeloma cells, growing the cells in a selection medium which does not support survival of the unfused cells, e.g., in HAT medium, screening the supernatants of the surviving hybridoma cells with recombinant PrP for the presence of antibodies to detect recombinant bovine PrP by an ELISA procedure and to detect native bovine Prp^{sc} by a conformation-sensitive ELISA procedure and isolating the positive cells. Positive hybridomas were selected and cloned twice by the limiting dilution method before the antibody was characterized and the epitope was mapped on a peptide library.

One peptide library that can be used is commercially available from Jerini Biotools (Berlin Germany). It consists of 104 spots with peptides of 13 amino acids, whereby the sequence of each peptide overlaps with 11 amino acids of the foregoing peptide.

An immunizing amount of a recombinant bovine prion protein is from about 50 to 100 μg. It is administered dissolved in an appropriate solvent, e.g., PBS and Freund's adjuvant several times, e.g., three times, subcutaneously followed by an intraperitoneal and an intravenous injection ultimately prior to spleen removal.

Appropriate myeloma cells are for example P3X63Ag8U.1 and are deposited and available under ATCC CRL 1597.

Recovering spleen cells and fusion conditions follow standard procedures, for example, as described by Kennett (Kennett, R. H. Fusion centriugation of cells suspended in polyethylene glycol. In Monoclonal antibodies. Hybridomas: a new dimension in biological analysis. (New York: Plenum Press) (1980) pp. 365-367).

An immunological detection of prion disease, especially disease-specific PrP^{sc} protein in a sample comprising, e.g., biological material of an animal or human is detected, comprises incubating a suitable affinity ligand, e.g., an appropriate antibody, with the sample under condition suitable for forming an antibody-prion protein complex which conditions are well known to those skilled in the art. In one aspect, a specific monoclonal antibody according to the invention is able to detect Prp^{sc} without prior protease-digestion of the tissue specimen to be examined. In one aspect, the affinity ligand, antibody, is modified in that it has conjugated to it a template.

The biological material can be insoluble or soluble in buffer or body fluids. It can be derived from any part of the body, e.g, from the brain or the tissue sections, in which case it is used in form of a homogenate, or any body fluid, e.g., cerebrospinal fluid, urine, saliva or blood. In the case of body fluids, fluid-resident cells, e.g., white blood cells in the case of blood expressing PrP can be purified and analyzed either in immunohistochemistry or as a homogenate.

Tissue homogenates and body fluids include, e.g., biopsy of brain, lymph nodes, spleens, tonsils, peripheral nerves, cerebrospinal fluids, urine, platelets or white blood cells.

Template Nucleic Acid Molecules

The detector molecules of the present invention comprise an antibody attached to a template nucleic acid molecule which is replicable with a nucleic acid polymerase.

The template nucleic acid molecules comprise any polynucleotide sequence, or a fragment thereof, which is replicable with a nucleic acid polymerase such as a DNA polymerase or RNA polymerase. In one embodiment, the RNA polymerase is a Q-beta replicase from bacteriophage Q-beta (Haruna and Spiegelman 1965 Science 150:884-886) or brome mosaic virus replicase (March et al., 1987 in “Positive Strand RNA Viruses” Alan R. Liss, N.Y.). In another embodiment, the RNA polymerase is a modified form of Q-beta replicase, such as Q-Amp (Q-RNA, Inc., New York, N.Y.).

The template nucleic acid molecule can be double-stranded or single-stranded DNA, or fragments thereof. The template nucleic acid molecule can be RNA, DNA/RNA hybrids, related molecules or fragments thereof. The template molecule can be a nucleic acid molecule based on an alternative backbone or including alternative bases, whether derived from natural sources or synthesized.

The template nucleic acid molecule can be derivative nucleic acid molecules, such as peptide nucleic acids (PNAs), and non-nucleic acid molecules including phosphorothioate, phosphotriester, phosphoramidate, and methylphosphonate molecules, (PC Zamecnik, et al., 1978 Proc. Natl. Acad. Sci. 75:280284; PC Goodchild, et al., 1986 Proc. Natl. Acad. Sci. 83:4143-4146; F Eckstein 1991 in: “Oligonucleotides and Analogues”, IRL Press, New York; M J Gait 1984 in: “Oligonucleotide Synthesis” IRL Press, Oxford, England; and Egholm 1992 in: “Innovative and Perspectives in Solid Phase Synthesis” pp 325-328 or U.S. Pat. No. 5,539,082).

It is understood that complementary base-pairing of individual base pairs generally follows Chargaff's Rule wherein an adenine pairs with an uracil (or thymine if DNA) and guanine pairs with cytosine. However, there are modified bases that account for unconventional base-pairing. A modified nucleic acid is understood to mean herein a DNA or RNA nucleic acid molecule that contains chemically modified nucleotides. The term “nucleic acid analogue” is understood herein to denote non-nucleic acid molecules such as “PNA” and morpholino that can engage in base-pairing interactions with conventional nucleic acids. These modified bases and nucleic acid analogues are considered to be within the scope of the instant invention. For example, nucleotides containing deazaguaine and uracil bases can be used in place of guanine and thymine, respectively, to decrease the thermal stability of probes. Similarly, 5-methyl-cytosine can be substituted for cytosine in complexes if increased thermal stability is desired. Modification to the sugar moiety can also occur and is embraced by the present invention. For example, modification to the ribose sugar moiety through the addition of 2′-O-methyl groups which can be used to reduce the nuclease susceptibility of RNA molecules. Modifications occurring with different moieties of the nucleic acid backbone are also within the scope of this invention. For example, the use of methyl phosphate, methyl phosphonate or phosphorothioate linkages to remove negative charges from the phosphodiesters backbone can be used.

Examples of suitable DNA templates are midi-variant DNA (MDV DNA), minivariant DNA (MNV DNA), MNV-API DNA, MNVUP DNA, MNVLO DNA, and combinations thereof.

The DNA sequence encoding MDV (SEQ ID NO:1) is:

5′ GGGGACCCCCCGGAAGGGGGGACGAGGTGCGGGCACCTCGTACGGGA
GTTCGACCGTGACGAGTCACGGGCTAGCGCTTTCGCGCTCTCCCAGGTGA
CGCCTCGTGAAGAGGCGCGACCTTCGTGCGTTTCGGCGACGCACGAGAAC
CGCCACGCTGCTTCGCAGCGTGGCCCCTTCGCGCAGCCCGCTGCGCGAGG
TGACCCCCGAAGGGGGGTTCCCCA 3′

The DNA sequence encoding MNV (SEQ ID NO:2) is:

5′ GGGTTCATAGCCTATTCGGCTTTTAAAGGACCTTTTTCCCTCGCGTA
GCTAGCTACGCGAGGTGACCCCCCGAAGGGGGGTGCCCC 3′

The DNA sequence encoding MNV-API (SEQ ID NO:3) is:

5′ GGGTTCATAGCCTATTCGGCTTCGCGCATGGGAATTTGAGGGACGAT
GGGGAAGTGGGAGCGCGTTTTAAAGGACCTTTTTCCCTCGCGTAGCTAGC
TACGCGAGGTGACCCCCCGAAGGGGGGTGCCCC 3′

The DNA sequence encoding MNVUP (SEQ ID NO:4) is:

5′ GGGTTCATAGCCTATTCGGCTTCGCGCCCGTTTATAATACTTAGTGA
GCGCGTTTTAAAGGACCTTTTTCCCTCGCGTAGCTAGCTACGCGAGGTGA
CCCCCCGAAGGGGGGTGCCCC 3′

The DNA sequence encoding MNVLO (SEQ ID NO: 5) is:

5′ GGGTTCATAGCCTATTCGGCTTCGCGCCCCTGGGGTTTGCCTCAGGA
GCGCGTTTTAAAGGACCTTTTTCCCTTGCGTAGCTAGCTACGCGAGGTGA
CCCCCCGAAGGGGGGTGCCCC 3′

In one aspect of the present invention, the template is an RNA molecule. For example, RQ 11+12.

The RNA sequence for RQ11+12 (SEQ ID NO:6) is:

5′ GGGGUUUCCAACCGGAAUUUGAGGGAUGCCUAGGCAUCCCCCGUGCG
UCCCUUUACGAGGGAUUGUCGACUCUAGUCGACGUCUGGGCGAAAAAUGU
ACGAGAGGACCUUUUCGGUACAGACGGUACCUGAGGGAUGCCUAGGCAUC
CCCCGCGCCGGUUUCGGACCUCCAGUGCGUGUUACCGCACUGUCGAC
CC 3′

In one aspect of the present invention, the template is the DNA version of RQ 11+12. The DNA sequence for RQ11+12 (SEQ ID NO: 7) is:

5′ GGGGTTTCCAACCGGAATTTGAGGGATGCCTAGGCATCCCCCGTGCG
TCCCTTTACGAGGGATTGTCGACTCTAGTCGACGTCTGGGCGAAAAATGT
ACGAGAGGACCTTTTCGGTACAGACGGTACCTGAGGGATGCCTAGGCATC
CCCCGCGCCGGTTTCGGACCTCCAGTGCGTGTTACCGCACTGTCGAC
CC 3′

The template nucleic acid molecules comprise polynucleotide sequences identical or similar to the nucleic acid molecules disclosed herein (e.g., SEQ ID NOS: 1-7), so long as they are replicable with a nucleic acid polymerase. In one embodiment, the template molecules exhibit sequence similarity with any of the sequences disclosed in SEQ ID NOS: 1-7, such as template molecules having at least 60% to 99.9% sequence similarity. The template molecules can include mutations such as deletions, insertions, additions, or truncations.

The length of the template nucleic acid molecule has no absolute length requirements. The length can range from about 20 to about 10,000 nucleotides. In one embodiment, the length of the template is about 20 to 50 nucleotides. One of ordinary skill in the art will be able to determine the appropriate length of nucleotide sequence to employ for the present invention.

The template nucleic acid molecules can be linked or labeled with a detectable marker. Examples of a detectable marker include, but are not limited to, a radioisotope, a fluorescent compound, a bioluminescent compound, a chemiluminescent compound, a metal chelator or an enzyme. Technologies for generating labeled nucleic acid molecules are well known (Sambrook et al., 1989 in “Molecular Cloning”).

Linker Molecules

The template nucleic acid molecule can be attached to the affinity ligand (e.g., antibody) using a linker molecule. In one embodiment, the template molecule and antibody are attached to each other via biotin-avidin interaction. Accordingly, the template nucleic acid molecule is linked with biotin (or derivative thereof), and the antibody is linked with avidin (or derivative thereof). The template nucleic acid molecule can be attached to the antibody before or after the protein detection assay is performed. This method of conjugation is well known to those skilled in the art. Other suitable methods are also appreciated by the skilled artisan for conjugating a nucleic acid molecule onto an antibody.

As mentioned above, the templates can be amplified using Q-Amp. Q-Amp is derived from Q-beta replicase. Q-beta replicase can be isolated and purified from Q-beta bacteriophage. Q-Amp is derived from the Q-beta replicase and comprises eukaryotic elongation factor Ts (Ef-Ts), eukaryotic elongation factor Tu (Ef-Tu), S1 nuclease, and a Replicase component. (Q-Amp is available from Q-RNA, Inc, New York, N.Y.) Q-Amp recognizes nucleic acid templates and can amplify them exponentially, e.g., up to one billion-fold, in fifteen minutes under isothermal conditions. Templates for Q-Amp can contain sequence insertions that may have specific functional applications. It should be appreciated by those skilled in the art, that replicases other than Q-Amp and Q-beta replicase may be used and are considered to be within the scope of this invention.

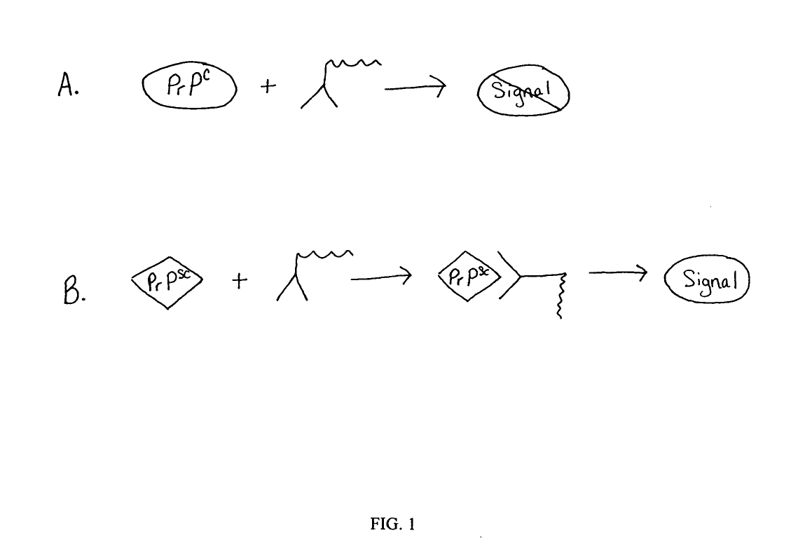

FIG. 1 illustrates the principle underlying the methods of the present invention. In FIG. 1a, native prion protein (PrP^{C}) is admixed with an antibody specific for the scrapies form of prion (PrP^{SC}). The antibody has conjugated to it a template represented by the squiggly line. As depicted in FIG. 1a, the antibody does not react with or bind to the native prion, hence there will be no antibody-antigen complex resulting in no signal. However, FIG. 1b depicts a scrapies form prion admixed with an antibody specific for PrP^{sc}. A complex is formed resulting in the formation of a signal.

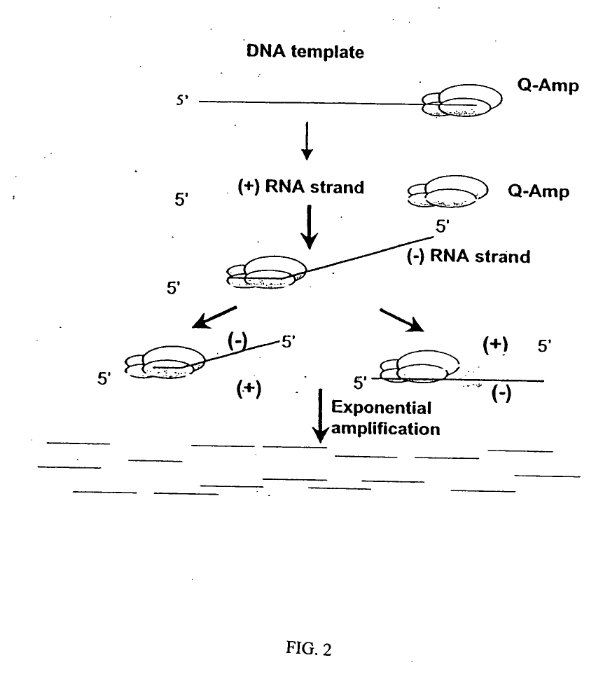

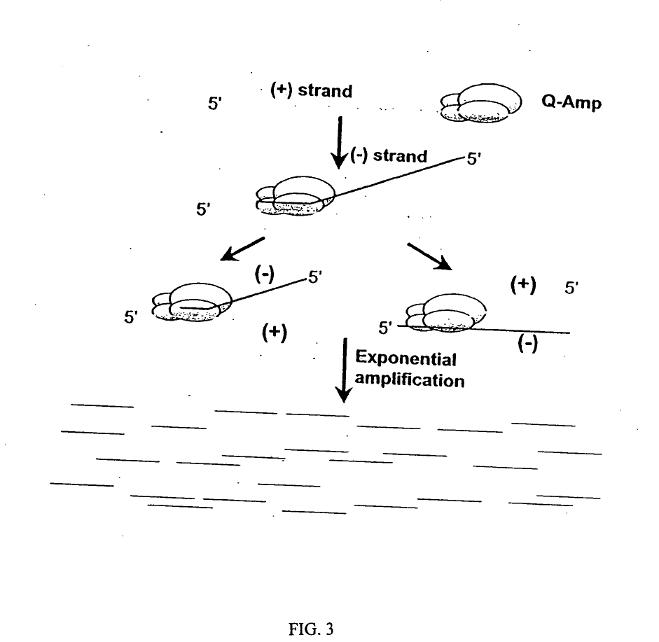

Detection of an antibody-antigen complex can be accomplished using several techniques. In the present invention, detection of an antibody-antigen complex is indicative of the presence of one or more target molecules within a given sample. In one aspect of the present invention, the template facilitates a detectable signal. As indicated above, the antibody of the present invention is modified comprising one or more template molecules. These templates are substrates for Q-Amp and are replicated under suitable conditions which are known to those skilled in the art. As mentioned above, Q-Amp can amplify a template a billion-fold plus in a matter of minutes. For example, if the template is a DNA molecule (like one described above), then Q-Amp can amplify this DNA template a billionfold plus in a short period of time. See FIG. 2. The nascent DNA single-strands will comprise both (−) and (+) strands that under proper conditions the complementary strands will hybridize forming double-stranded DNA molecules. These newly synthesized doublestranded DNA molecules can be detected using, e.g., an intercalating agent such as ethydium bromide which will emit a signal when intercalated within a double-stranded DNA. Other classes of nucleotides can serve as templates such as RNA. See FIG. 3.

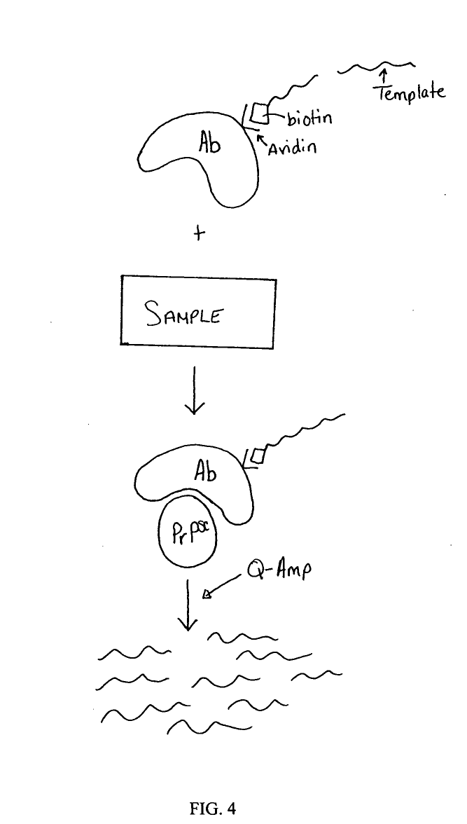

FIG. 4 depicts one embodiment of the present invention. This embodiment includes an antibody specific for PrP^{sc} which is conjugated to a template via an avidin-biotin interaction. The conjugated antibody is admixed with a sample putatively containing PrP^{sc}. The conditions are suitable for antibody-antigen complex formation. The complex is separated from free antibody. Q-Amp is admixed with isolated complex to form nascent nucleic acid molecules. These nascent molecules can be detected using methods described herein as well as those well known to those skilled in the art.

In order to minimize or eliminate false positives, the antibody-antigen complex has to be isolated from the free (unbound) antibody. There are several well accepted methods for effectuating this isolation. For example, isolation can be realized by employing a size separation technique. The target (antigen) can be fixed to a substrate. Following a suitable period of incubation with a homogenous or heterogenous preparation of template containing antibody, a wash step can be performed eliminating unbound antibody. Thus, the remaining antibody is that which is bound to the target.

One skilled in the art will appreciate that there are other well known methods for producing a signal such as the use of a label that emits energy which can be detected by a suitable detector. For example, labeled nucleotide base precursors can be used in the replication reaction. For example, adenine can be labeled with a radioactive label or a fluorescence label, or any other suitable label that can be detected. As the nascent nucleic acid is being synthesized using the conjugated template, labeled nucleotides will be incorporated into the nascent nucleotide.

Since the present invention provides a rapid method for detecting a target that also does not require special laboratory equipment, the present invention is adaptable for use in clinical diagnostics assays as point of care (POC) “dip and read” type assays and automated ELISA tests.

The kit can be in any immuno-assay format, including immuno-stick (dip stick), an enzyme-linked immuno-sorbent assay (ELISA), a sandwich assay, a dot blot assay, a radioimmunoassay, a radioimmunoprecipitation, an immunofluorescent assay, an immunochromatographic assay, an immunofiltration assay, a latex bead agglutination assay, a biosensor assay, a western blot assay, an inhibition or competition assay, a simultaneous assay, or a countercurrent immuno-electrophoresis (CIEP) assay.

These immuno-assays can be performed sequentially or simultaneously. In these immuno-assays, the solid phase can be a polystyrene, nylon, nitrocellulose, cellulose acetate, chromatographic strip, latex bead or colloidal gold.

In one embodiment, the kit comprises the inventive modified detector molecule comprising the affinity ligand (e.g., antibody) attached to the template nucleic acid molecule (e.g., DNA) which is replicable with a nucleic acid polymerase (e.g., Q-beta replicase or Q-Amp replicase). In another embodiment, the kit comprises the affinity ligand (e.g., antibody) separate from the template nucleic acid molecule (e.g., DNA) which is replicable with a nucleic acid polymerase (e.g., Q-beta replicase or Q-Amp replicase). The kit can include Q-beta replicase or Q-Amp enzyme. The Q-beta replicase or Q-Amp enzyme can be supplied in a container which is separate from the modified detector molecule, or separate from the affinity ligand and template nucleic acid molecule. Alternatively, the Q-beta replicase or Q-Amp enzyme is supplied with the modified detector molecule, or with the template nucleic acid molecule, as a liposome-encased reactant. The kit can also include a capture antibody which binds the target protein. The capture antibody can be bound to a solid support (solid phase). The kit can include rNTPs and/or NTPs. The rNTPs and/or NTPs can be labeled or linked with a detectable marker The kit can include appropriate buffers and reagents.

In one embodiment, a kit is describe for performing the methods of the present invention. The kit comprises an affinity ligand specific for and having affinity to a target molecule. In one aspect, the affinity ligand is an antibody. In a particular aspect, the affinity ligand has conjugated to it a template molecule. The template can be a nucleic acid molecule. Specifically, the template can be DNA, RNA, or a modification thereof. Examples of a suitable template are MDV, MNV, MNV-AP1, MNVUP, MNVLO, RQ11+12, fragments and derivatives thereof. Appropriate reagents such as buffers known to those skilled in the art can be supplied in the kit as well. The kit also comprises a replicase activity. An example of a suitable replicase is Q-Amp. Instructions can also be included within the kit.

### EXAMPLES

The following examples are meant to further enhance the understanding of the invention and are in no way meant to limit the invention.

### Example 1

Immunoglobulin ELISA-Qamp Protocol

I. Coat with Capture Antibody:

1. Dilute the purified mouse IgG, Whole Molecule (Pierce, cat. no. 31204), to 334 fm/ml (2 μg/ml, 10 fm/30 μl) in coating buffer (PBS or 0.1 M NaHCO3, pH 8.6). and to prepare 10×serial dilutions of mouse IgG (from 10 fM/30 μl to 0.1 aM/3011).

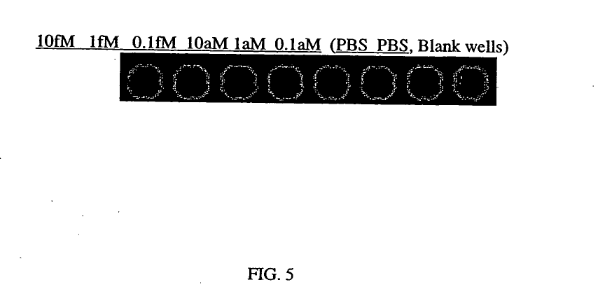

2.Add 30 μl per well to an enhanced protein-binding ELISA strips (e.g., Nunc TopYieid Strips, cat. no. 24-8909). See FIG. 5.

3. Shake plate to ensure all wells are covered by capture antibody solution.

4 Seal plate to prevent evaporation. and incubate for overnight at 4° C. (or 1 hour at 37° C.).

5. Bring the plate to RT, remove the capture antibody solution. Wash the plate 3× with PBS/Tween*. For each wash, wells are filled with 200 μl PBS/Tween and allowed to stand at least 1 minute prior to aspirating.

II. Blocking:

1. Block the plate with 200 μl blocking buffer*per well.

2.Cover the plate and incubate for overnight at 4° C. or at room temperature for 60 minutes.

3. Wash the plate 3× with PBS/Tween, as in Section I, Step 4, of this protocol.

III. Incubation with Detection Antibody:

1. Dilute biotinylated anti-mouse IgG (Pierce, cat. no. 31800) in 1:10 blocking buffer to 0.05-0.20 μg/ml (10-40 fm per 30 μl)

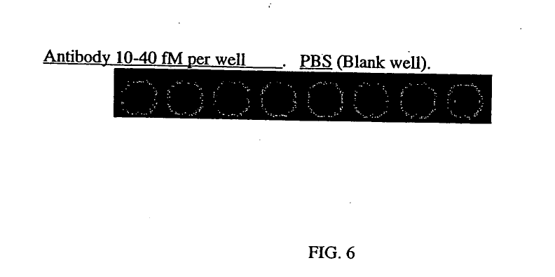

2. Add 30 μl per well. See FIG. 6.

3. Cover the plate and incubate at room temperature for 1 hour.

4. Wash the plate 6× with PBS/Tween,

IV. Add Neuroavidin-BiotinDNA-MNVII mix):

1. Dilute BiotinDNA-MNV11 (stock solution at 5 pM/μl) to 0.2 pM/μl in 10 mM tris.HCl, pH 7.4, 150 mM NaCl. (100%1)

2. Dilute Neuroavidin to 0.2 pM/μl in 10 mM tris.HCl, pH 7.4, 150 mM NaCl. (100 μl)

3. Mix and vortex. Incubate at RT 30-60 min. Solution may be stored for several weeks at 4 C.

4. Dilute Neuroavidin-BiotinDNA MNV11 mix to 2-10 fM/30 ml in 1:10 blocking buffer. Add 30 μl per well.

5. Cover the plate and incubate at room temperature for 60 minutes.

6 Wash the plate 8× with PBS-Tween, 2× with H₂O.

V. Add Qbeta replicase mix and Develop:

1. Dilute Qbeta replicase to 0.09-0.18 mg/ml (buffer 1×G, 0.5 mM NTP, 1.5 μg propidium iodide/ml).

2. Add 30 μl per well.

3. Incubate at 30-37 C (20-30 min).

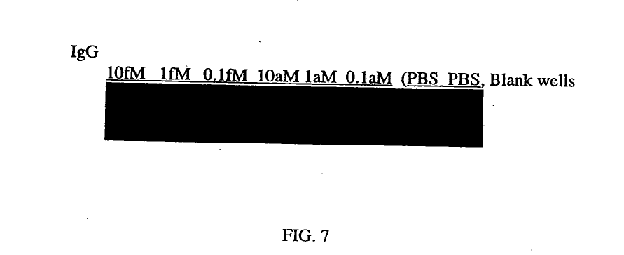

4. Transfer to UV box. Take a digital picture (yellow filter), See FIG. 7.

High backgrounds in blank wells or poor consistency of replicates can be overcome by increasing the stringency of washes and optimizing the concentration of detection antibody. For example, during washes, the wells can be soaked for 1 minute intervals. Moreover, lower concentrations of detecting antibody and bioDNAMNV11 or more washes can reduce background

Reagents:

| | |
|---|---|
| Tris-HCI, pH 7.4, | 1M |
| MgCl₂, | 1M |
| DTT, | 0.1M |
| NTP | 25 mM |
| Propidium | 0.1 μg/ml |
| Qbeta replicase | 4 mg/ml |
| H₂O/Sigma | |

Buffer 5×G:(1 ml): 400%1 Tris-HCl
50 μl MgCl₂
50 μl DTT
500 μl H₂O
   *SOLUTIONS
   Coating Buffer PBS Solution
   PBS, pH 7.2-7.4 NaCl 80.0 g
   Na2HPO 411.6 g
   PBS/Tween KH2PO4 2.0 g
   PBS KCl 2.0 g
   Tween-20 0.05% ddH2O to 10 L
   A
   Blocking Buffer
   PBS
   Nonfat dry milk 5%-10%, DNA herring sperm (SIGMA, cat. D3159-10G) 10 mg/ml.

High backgrounds in blank wells or poor consistency of replicates can be overcome by increasing the stringency of washes and optimizing the concentration of detection antibody and avidin-biotinDNA. For example, during washes, the wells can be soaked for ˜2-5 minute intervals. Moreover, lower concentrations of detecting antibody or more washes after the avidin-biotinDNA stage can reduce background.

The comparison of the sensitivity of the standard ELISA detection and ELISA-Qamp.

I. Standard ELISA protocol.

1. Mouse IgG 40 fM

3.Neuroavidin_Biotin-Horseradish Peroxidase (1:50000) mix (1:1) (50 μl).

4. Detection: Turbo TMB-ELISA (50 μl), 20 min.

2. Biotinylated anti-Mouse IgG antibody.

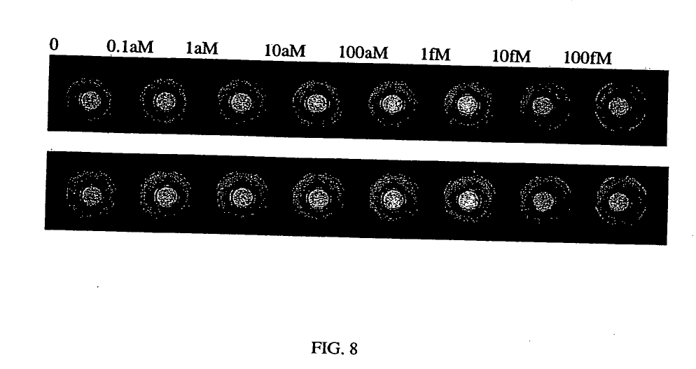

See FIG. 8.

II. ELISA-Qamp protocol.

1. Mouse IgG 40 fM

3. Neuroavidin_biotin-dMNv11 (1:1), 4 fM (30 l—tI)

4. Detection: Qbeta replicase (Qbeta repl. 2.4 μg/30 μl 0.75 mM NTP, 20 mM Mg++. 15 min

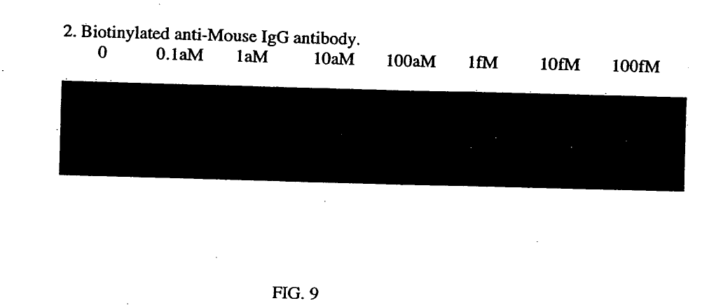

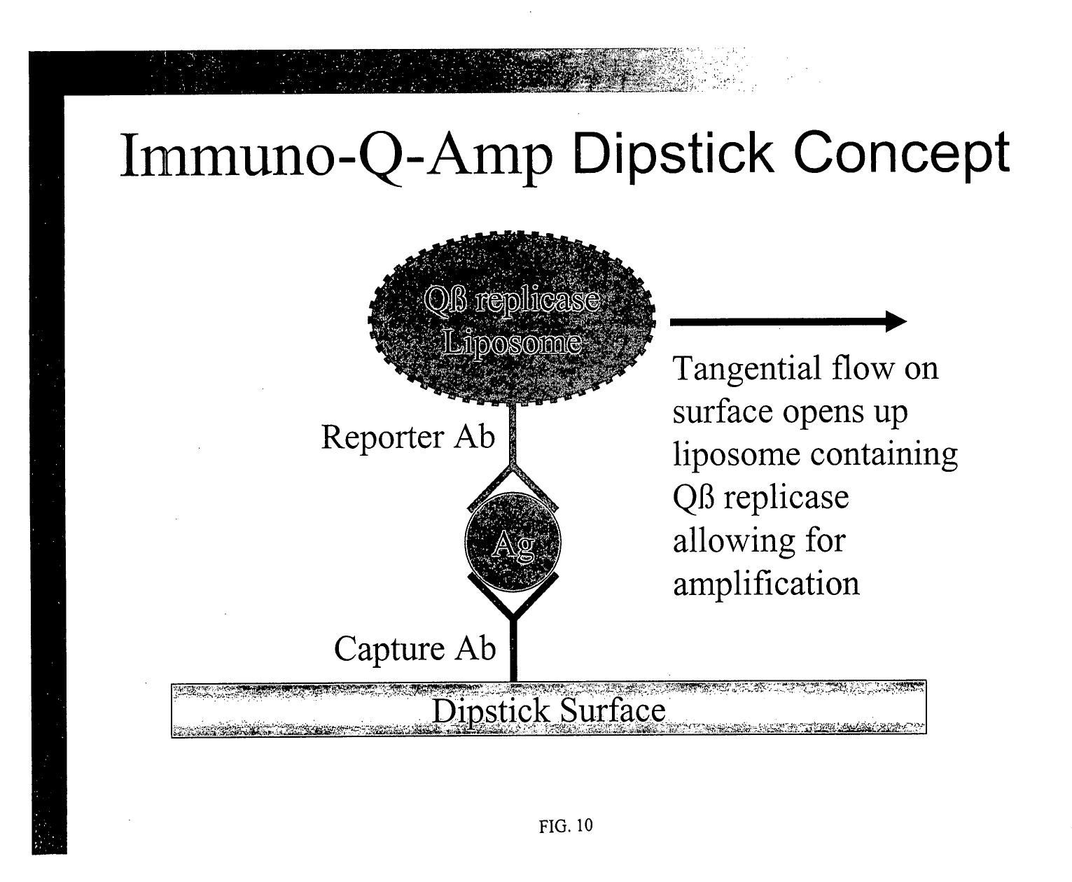

See FIG. 9.

### Example 2

The following provides a description of methods for detecting the presence or absence of a tau protein in a sample, using the protein detector molecule of the present invention. In this example, two different blocking reagents are compared.

Materials

Capture antibody: anti-TAU antibody MN1010 (Pierce, #MN1010).

Reporter antibody: anti-TAU, biotinylated antibody MN-1000 (Pierce, #MN1000B).

Blocking reagents: Superblock and Startingblock solutions (Pierce, #37515 and 37538, respectively).

Linker molecules: Avidin and neutravidin (Pierce).

TAU-412 target protein and Q-beta enzyme were prepared at Q-RNA, Incorporated (New York, N.Y.).

Nucleotide Triphosphates: ATP,CTP,GTP and UTP were purchased from Fermentas.

Biotin-MNV template DNA was purchased from Integrated DNA Technologies, Inc. 96-well Microfluor-2 plates (#14-245-176) and Sealant Tape were purchased from Fisher Corp.

PBS-Tween in powder form, Tris buffer (1M) pH 7.4, sodium chloride (5M solution) and molecular grade water were purchased from Sigma Chemicals.

Syber Green-1 was obtained from Molecular Probes.

Preparation of Buffers and Reagents:

DNA Templates: Avidin or Neutravidin-Biotin-dMNV (plus) Conjugate:

Tris buffer was diluted to 10 mM and sodium chloride was added to obtain a final concentration of 150 mMNaCl.

Avidin or neutravidin stock solutions: 1 mg of Avidin was dissolved in 1 ml Tris-HCl buffer, pH 7.4. Neutravidin (10 mg) was dissolved in 1 ml molecular grade water and 2 ul of this solution was added to 1.67 ml Tris NaCl Buffer

Avidin and Neutravidin working solutions: Avidin and neutravidin were diluted to 0.2 pm/ul by adding 2.69 ul of stock solution to 100 ul final volume in 10 mM Tris-HCl, pH 7.4, 150 mM NaCl.

Biotin MNV solution: Biotin-DNA-MNV11 (stock solution at 5 pm/ul) was diluted to 0.2 pm/ul by adding 5.3 to 100 ul final volume in 10 mM Tris-HCl, pH 7.4, 150 mM NaCl.

Reaction: Biotin-dMNV11(plus) and avidin/neutravidin, was vortexed and incubated at room temperature for 30 minutes. The solution was stored at 4 degrees C.

Coating Buffer:

0.05M Sodium Carbonate buffer pH 9.6 was prepared as follows:

0.05M Sodium Carbonate Solution

MW: 106

5.3 g/liter for 0.05M solution

Weigh: 0.265 g, dissolve in 50 ml de-ionized water

0.05M Sodium Bicarbonate Solution

MW: 84

4.2 g/liter for 0.05M solution

Weigh: 0.21 g, dissolve in 50 ml de-ionized water

Added carbonate solution (about 21 ml) to bicarbonate to obtain pH of 9.6

Stored at 4 degrees C.

PBS-Tween:

PBS-Tween powder was dissolved in molecular grade water (l packet per liter).

5×G Buffer:

400 ul Tris-HCl, pH 7.4 (1M Stock)

50 ul MgCl₂ (1M Stock)

50 ul DTT (0.1M Stock)

500 ul molecular grade water

4×SYBR Green I:

10 mM Tris-HCl, pH 8.0

1 mM EDTA

Enzyme AMP Mix Cocktail (for 2 plates) was prepared as follows:

| | |
|---|---|
| Number of wells | 36 |
| Assay volume (ul) | 50 |
| [QB] ug/ul | 4.3 |
| [QB] ug/well | 2.5 |

TABLE 1

| AMP Mix Setup | Volume (ul) | |
|---|---|---|
| Molecular grade water | 1422 | |
| NTP mix | 100.8 | |
| (ATP, GTP, CTP and | | |
| UTP) | | |
| 5XG buffer | 432 | |
| 4x SYBR Green I | 180 | |
| QB | 25.2 | |
| Total | 2160 | <= allows 20% volume for overage |

Methods:
Plate Coating:
Coating solution: 100 uL of Anti-TAU MN 1010 (200 ug/mL) was diluted to 4 mL in coating buffer to obtain a coating solution of 5 ug/mL.
Coating: 100 uL of this solution was added to each well as per Table 1 and plate sealed with sealant tape. Coating was done either at 37 degrees C. for one hour or overnight at 4 degrees C.

Washing: The coated plate was washed three times with 230 uL per well of PBS-Tween.

TABLE 2

| | 1 | 2 | 3 | 4 | 5 | 6 | 7 | 8 | 9 | 10 | 11 | 12 |
|---|---|---|---|---|---|---|---|---|---|---|---|---|
| A | X | X | X | X | X | X | X | X | X | X | X | X |

| | | | | | | |
|---|---|---|---|---|---|---|
| B | X | Coat | Coat | Coat | X | X |
| C | X | Coat | Coat | Coat | X | X |
| D | X | Coat | Coat | Coat | X | X |
| E | X | Coat | Coat | Coat | X | X |
| F | X | Coat | Coat | Coat | X | X |
| G | X | Coat | Coat | Coat | X | X |
| H | X | No coating | No coating | No coating | X | X |

Blocking

200 ul of Blocking solution (Superblock or Starting Block) was added to each well, the plate was sealed, incubated at 37 degrees C. for one hour and washed.

TABLE 3

| | | Superblock | | | Starting block | | | Superblock | | | | |
|---|---|---|---|---|---|---|---|---|---|---|---|---|
| | 1 | 2 | 3 | 4 | 5 | 6 | 7 | 8 | 9 | 10 | 11 | 12 |
| A | X | X | X | X | X | X | X | X | X | X | X | X |

| | | | | | | |
|---|---|---|---|---|---|---|
| B | X | block | block | block | X | X |
| C | X | block | block | block | X | X |
| D | X | block | block | block | X | X |
| E | X | block | block | block | X | X |
| F | X | block | block | block | X | X |
| G | X | No blocking | No blocking | No blocking | X | X |
| H | X | No blocking | No blocking | No blocking | X | X |

Target Binding
Target solution: TAU-412 (1 mg/mL) was diluted in PBS-Tween to obtain 100 ng/mL, by adding 2 uL of TAU to 20 mL PBST.
Binding: 100 uL of the binding solution was added to the appropriate wells as indicated in the following Table. Plate was incubated for one hour at 37 degrees C. for one hour and washed.

Tau412 Sequence:

Acc# AY730549 gi# 52421758
“MAEPRQEFEVMEDHAGTYGLGDRKDQGGYTMHQDQEGDTDAGLKESPPQ
TPTEDGSEEPGSETSDAKSTPTAEAEEAGIGDTPSLEDEAAGHVTQARMV
SKSKDGTGSDDKKAKGADGKTKIATPRGAAPPGQKGQANATRIPAKTPPA
PKTPPSSGEPPKSGDRSGYSSPGSPGTPGSRSRTPSLPTPPTREPKKVAV
VRTPPKSPSSAKSRLQTAPVPMPDLKNVKSKIGSTENLKHQPGGGKVQII
NKKLDLSNVQSKCGSKDNIKHVPGGGSVQIVYKPVDLSKVTSKCGSLGNI
HHKPGGGQVEVKSEKLDFKDRVQSKIGSLDNITHVPGGGNKKIETHKLTF
RENAKAKTDHGAEIVYKSPVVSGDTSPRHLSNVSSTGSIDMVDSPQLATL
ADEVSASLAKQGL”

TABLE 4

| | 1 | 2 | 3 | 4 | 5 | 6 | 7 | 8 | 9 | 10 | 11 | 12 |
|---|---|---|---|---|---|---|---|---|---|---|---|---|
| A | X | X | X | X | X | X | X | X | X | X | X | X |

| | | | | | | |
|---|---|---|---|---|---|---|
| B | X | Test | Test | Test | X | X |
| C | X | No Tau | No Tau | No Tau | X | X |
| D | X | No Reporter | No Reporter | No Reporter | X | X |
| E | X | No Template | No Template | No Template | X | X |
| F | X | No Tau No | No Tau No | No Tau No | X | X |
| | | Template | Template | Template | | |
| G | X | No block No | No block No | No block No | X | X |
| | | Tau | Tau | Tau | | |
| | | No Template | No Template | No Template | | |
| H | X | Enzyme amp | Enzyme amp | Enzyme amp | X | X |
| | | mix only | mix only | mix only | | |

Reporter Antibody

Reporter solution: Biotin MN1000 was diluted 1 to 6000 by adding 2 uL to 12 mL PBST and 50 uL of the reporter solution was added to appropriate wells as shown in the Table, plate incubated at 37° C. for one hour and washed with PBS-Tween

TABLE 5

| | 1 | 2 | 3 | 4 | 5 | 6 | 7 | 8 | 9 | 10 | 11 | 12 |
|---|---|---|---|---|---|---|---|---|---|---|---|---|
| A | X | X | X | X | X | X | X | X | X | X | X | X |

| | | | | | | |
|---|---|---|---|---|---|---|
| B | X | Reporter | Reporter | Reporter | X | X |
| C | X | Reporter | Reporter | Reporter | X | X |
| D | X | Reporter | Reporter | No Reporter | X | X |
| E | X | Reporter | Reporter | Reporter | X | X |
| F | X | Reporter | Reporter | Reporter | X | X |
| G | X | Reporter | Reporter | Reporter | X | X |
| H | X | No Reporter | No Reporter | No Reporter | X | X |

Template Binding
Template dilution: Avidin-Biotin-MNVp DNA Template (3.76×10¹² molecules/ul) was diluted as follows to obtain the working solution of 1×10⁴ molecules/ul.
2 uL of stock into 200 ul PBS Tween to make 100× dilution. (1×10⁹ molecules/ul)
2 ul of 100× into 2 ml PBS Tween (1×10⁶ molecules/ul)
200 ul of 1×10⁶ molecules/ul into 2 ml PBS Tween (1×10⁵ molecules/ul)
200 ul of 1×10⁵ molecules/ul into 2 ml PBS Tween (1×10⁴ molecules/ul)

25 uL of template solution (1×10⁴ molecules/ul) was added to appropriate wells as per the following table, incubated for 30 minutes at room temperature. The plate was washed and placed on a cold block.

TABLE 6

| | 1 | 2 | 3 | 4 | 5 | 6 | 7 | 8 | 9 | 10 | 11 | 12 |
|---|---|---|---|---|---|---|---|---|---|---|---|---|
| A | X | X | X | X | X | X | X | X | X | X | X | X |

| | | | | | | |
|---|---|---|---|---|---|---|
| B | X | Template | Template | Template | X | X |
| C | X | Template | Template | Template | X | X |
| D | X | Template | Template | Template | X | X |
| E | X | No template | No template | No template | X | X |
| F | X | No template | No template | No template | X | X |
| G | X | No template | No template | No template | X | X |
| H | X | No template | No template | No template | X | X |

The DNA sequence of MNVplus (SEQ ID NO. 8) is:

5′GGGTTCATAGCCTATTCGGCTTTTAAAGGACCTTTTTCCCTCGCGTAG
CTAGCTACGCGAGGTGACCCCCCGAAGGGGGGTGCCCC 3′

The DNA sequence of MNVminus (SEQ ID NO. 9) is:

5′GGGGCACCCCCCTTCGGGGGGTCACCTCGCGTAGCTAGCTACGCGAGG
GAAAAGGTCCTTTAAAAGCCGCCTAGGCTATGAACCC 3′

Q-AMP Replicase Reaction:
To the cold plate was added 50 uL of Enzyme-AMP mix cocktail. Fluoroskan-2 fluorometer, was set at 37 degrees C. and in the kinetic mode. Fluorescence (485 nm ex/520 em) was recorded every 90 seconds for 45 minutes.
Data Analysis

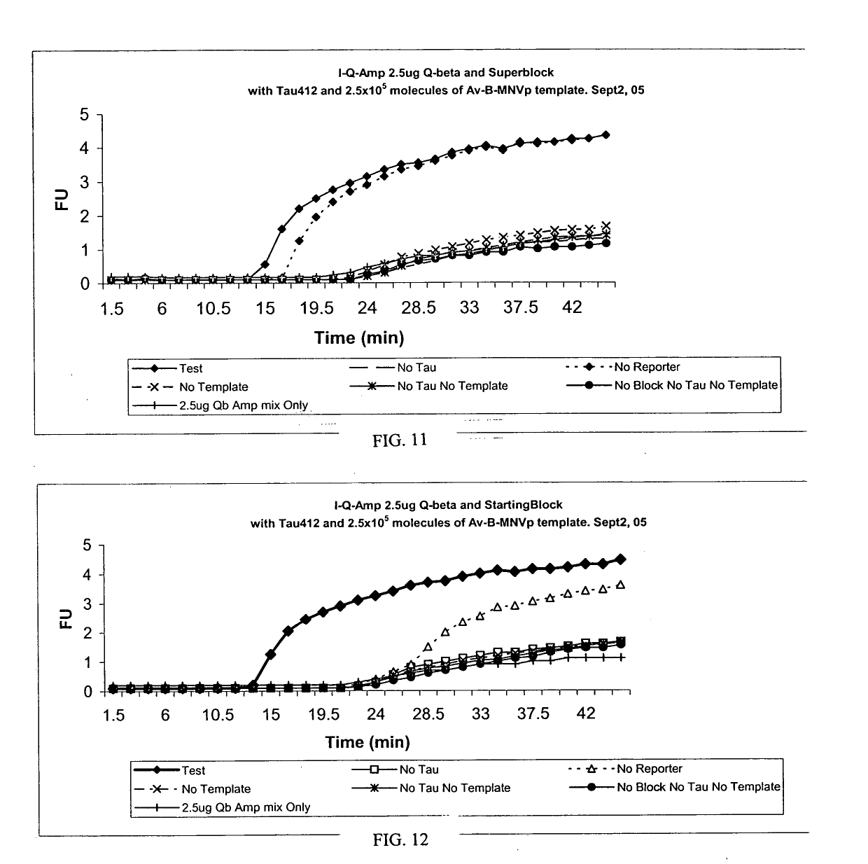

Fluorescent Units (FU) of replicate wells were averaged for each time point and a plot was constructed with time against FU (avg) for test and controls (Superblock, FIG. 11; and Startingblock, FIG. 12) The results were expressed as the response time (RT) for each sample, obtained by extrapolating the linear phase of each curve and noting the point of intersection with x-axis.

TABLE 7

| sample | RT (Superblock blocking) | RT (starting block blocking) |
|---|---|---|
| Test | 15 min | 13.5 min |
| No reporter | 16.5 min | 25.5 min. |

Results show that use of Starting Block as blocking buffer gives a faster response time for test and slower for control.

The methods of the present invention can be performed using a capture antibody to bind with the target protein present in the sample to form a captured protein complex. The captured protein is subsequently reacted with a reporter antibody that binds the protein. The reporter antibody is not attached to the template DNA initially during this step of the detection method. The template DNA is subsequently added to bind to the reporter antibody thereby forming the modified detector molecule. The replicase enzyme and appropriate reactants (e.g., NTPS) are added to permit the replicase to replicate the template DNA thereby forming copies of RNA strands. The binding steps and the replicase reaction can be performed under normal pH (e.g., about pH 7), at a range of about 20-37° C., for about 20-25 minutes.

In this capture antibody embodiment, the modified detector molecule (or “protein detector molecule”) detects and reports the presence of human tau 412 protein, the method comprises the following steps. (1) A sample is contacted with an anti-tau capture antibody under conditions that are suitable for the capture antibody to form a captured protein complex (capture antibody/tau). The capture antibody is bound to a solid support. Unbound components in the sample can be removed by washing, aspiration, or other similar procedures. (2) The captured protein complex is contacted with an anti-tau reporter antibody under conditions that are suitable for the reporter antibody to bind with the captured protein complex to form a sandwich complex (capture antibody/tau/reporter antibody). The unbound reporter antibody can be removed by washing, aspiration, or other similar procedures. (3) The sandwich complex is contacted with the template DNA molecule under conditions that are suitable for the template DNA molecule to bind with the reporter antibody to form a replicable complex (capture antibody/tau/reporter antibody/template DNA). In this step, the binding of the template DNA to the reporter antibody forms the protein detector molecule. The unbound template DNA can be removed by washing, aspiration, or other similar procedures. (4) The replicable complex is reacted with a replicase enzyme, such as Q-beta replicase or Q-Amp replicase, under conditions that are suitable for the replicase enzyme to produce replicated RNA strands from the template DNA. The replicated RNA strands can be labeled with a detectable marker to permit reporting of the presence of the target protein.

Example 2 describes a capture antibody experiment. This experiment was performed to detect human tau protein in a sample using a capture antibody, and reporter antibody separate from the template DNA in the initial steps. The binding and replicase steps of the experiment were performed at pH 7.4, at 37° C., for 90 minutes. The experiment also compared two different commercially-available blocking reagents, Superblock™ and Startingblock™ (both from Pierce). The results (FIGS. 11 and 12) demonstrate that this method is highly sensitive, being capable of detecting 10 nano grams of human tau protein (equivalent to attomol concentration of protein) in fewer than 20 minutes, under isothermal conditions. The results also show this method is specific, because very little signal is detected in the samples lacking tau protein or template DNA.

The capture antibody embodiment described above is merely provided for illustrative purposes and is not intended to limit the scope of the present invention. The skilled artisan will appreciate there are many other ways to perform a protein detection method using the inventive protein detector molecule.

The capture antibody embodiment described above is merely provided for illustrative purposes and is not intended to limit the scope of the present invention. The skilled artisan will appreciate there are many other ways to perform a protein detection method using the inventive protein detector molecule.

Although the invention has been described with respect to various embodiments, it should be realized this invention is also capable of a wide variety of further and other embodiments within the spirit and scope of the appended claims.

What is claimed:

1. A composition for detecting a tau protein comprising a modified detector molecule having two ends, a first end capable of binding the tau protein and a second end comprising a single-stranded DNA template, wherein the template is capable of being replicated by an RNA polymerase.

2. The composition of claim 1, wherein the single-stranded DNA is selected from the group consisting of MDV DNA, MNV DNA, MNV-AP1 DNA, MNVUP DNA, MNVLO DNA, MNVplus DNA, MNVminus DNA, RQ 11+12 DNA, and fragments and derivatives thereof.

3. The composition of claim 1, wherein the single-stranded DNA is a positive strand or a negative strand.

4. The composition of claim 1, wherein the RNA polymerase is a Q-beta replicase.

5. The composition of claim 4, wherein the Q-beta replicase is a modified Q-beta replicase.

6. The composition of claim 5, wherein the modified Q-beta replicase is Q-Amp.

7. The composition of claim 1, wherein the single-stranded DNA is any single-stranded DNA capable of being replicated by Q-beta replicase.

8. The composition of claim 1, wherein the first end of the modified detector molecule is an antibody.

9. The composition of claim 8, wherein the antibody is a monoclonal antibody.

10. The composition of claim 1, wherein the tau protein is a misfolded tau protein.

11. The composition of claim 10, wherein the presence of a misfolded tau protein is indicative of a neurogenic disease.

12. The composition of claim 11, wherein the neurogenic disease is Alzheimer's.

13. The composition of claim 1, wherein the template is further attached to a detectable label.

14. The composition of claim 13, wherein the label is a fluorescent label.

15. The composition of claim 13, wherein the label is a radioactive label.

16. A composition for the detection of a tau protein comprising:
(a) an affinity ligand capable of binding to the tau protein;
(b) a single-stranded DNA template capable of being replicated by a RNA polymerase; and (c) a linker, wherein the linker links the affinity ligand to the template.

17. The composition of claim 16, wherein the single-stranded DNA is selected from the group consisting of MDV DNA, MNV DNA, MNV-AP1 DNA, MNVUP DNA, MNVLO DNA, MNVplus DNA, MNVminus DNA, RQ 11+12 DNA, and fragments and derivatives thereof.

18. The composition of claim 16, wherein the single-stranded DNA is a positive strand or a negative strand.

19. The composition of claim 16, wherein the replicase is a Q-beta replicase.

20. The composition of claim 19, wherein the replicase is a modified Q-beta replicase.

21. The composition of claim 20, wherein the modified Q-beta replicase is Q-Amp.

22. The composition of claim 16, wherein the single-stranded DNA is any single-stranded DNA capable of being replicated by Q-beta replicase.

23. The composition of claim 16, wherein the affinity ligand is an antibody.

24. The composition of claim 23, wherein the antibody is a monoclonal antibody.

25. The composition of claim 16, wherein the tau protein is a misfolded tau protein.

26. The composition of claim 25, wherein the presence of the misfolded tau protein is indicative of a neurogenic disease.

27. The composition of claim 24, wherein the neurogenic disease is Alzheimer's disease.

28. The composition of claim 16, wherein the template is further attached to a detectable label.

29. The composition of claim 28, wherein the label is a fluorescent label.

30. The composition of claim 28, wherein the label is a radioactive label.

31. The composition of claim 16, wherein the linker is a covalent bond.

32. The composition of claim 16, wherein the linker comprises biotin and Avidin or its derivatives.

33. A method for detecting the presence of a tau protein, comprising:
(a) contacting a sample to a modified detector molecule, such that a complex will be formed in the presence of tau protein, the modified detector molecule having two ends, a first end capable of binding to the tau protein, and a second detector end comprising a single-stranded DNA template, wherein the template is capable of being replicated by an RNA polymerase;
(b) contacting the complex formed in (a) with an RNA polymerase such that the DNA template will be replicated in the presence of the target; and
(c) detecting the replicated DNA template thereby indicating the presence of the tau protein.

34. The method of claim 33, wherein the single-stranded DNA is selected from the group consisting of MDV DNA, MNV DNA, MNV-AP1 DNA, MNVUP DNA, MNVLO DNA, MNVplus DNA, MNVminus DNA, RQ 11+12 DNA, and fragments and derivatives thereof.

35. The method of claim 33, wherein the single-stranded DNA is a positive strand or a negative strand.

36. The method of claim 33, wherein the RNA polymerase is a Q-beta replicase.

37. The method of claim 33, wherein the Q-beta replicase is a modified Q-beta replicase.

38. The method of claim 33, wherein the modified Q-beta replicase is Q-Amp.

39. The method of claim 33, wherein the single-stranded DNA is any single-stranded DNA capable of being replicated by Q-beta replicase.

40. The method of claim 33, wherein the first end of the modified detector molecule is an antibody.

41. The method of claim 40, wherein the antibody is a monoclonal antibody.

42. The method of claim 33, wherein the tau protein is a misfolded tau protein.

43. The method of claim 42, wherein the presence of the misfolded tau protein is indicative of a neurogenic disease.

44. The method of claim 43, wherein the neurogenic disease is Alzheimer's disease.

45. The method of claim 33, wherein the template is further attached to a detectable label.

46. The method of claim 45, wherein the label is a fluorescent label.

47. The method of claim 45, wherein the label is a radioactive label.

48. The method of claim 33, wherein the method is performed at ambient temperature.

49. The method of claim 33, wherein the method is performed under thermo-cycling conditions.

50. A method for detecting the presence of tau protein, comprising
(a) contacting a sample to an affinity ligand capable of binding tau;
(b) further contacting the sample and the affinity ligand with a single-stranded DNA template capable of being replicated by an RNA polymerase, such that a complex will form in the presence of the target;
(c) contacting the complex of (b) with an RNA polymerase such that the DNA template will be replicated in the presence of the target; and
(d) detecting the replicated DNA template thereby indicating the presence tau protein.

51. The method of claim 50, wherein the single-stranded DNA is selected from the group consisting of MDV DNA, MNV DNA, MNV-AP1 DNA, MNVUP DNA, MNVLO DNA, MNVplus DNA, MNVminus DNA, RQ 11+12 DNA, and fragments and derivatives thereof.

52. The method of claim 50, wherein the single-stranded DNA is a positive strand or a negative strand.

53. The method of claim 50, wherein the RNA polymerase is a Q-beta replicase.

54. The method of claim 53, wherein the Q-beta replicase is a modified Q-beta replicase.

55. The method of claim 54, wherein the modified Q-beta replicase is Q-Amp.

56. The method of claim 50, wherein the single-stranded DNA is any single-stranded DNA capable of being replicated by Q-beta replicase.

57. The method of claim 50, wherein the affinity ligand is an antibody.

58. The method of claim 57, wherein the antibody is a monoclonal antibody.

59. The method of claim 50, wherein the tau protein is a misfolded tau protein.

60. The method of claim 59, wherein the presence of the misfolded tau protein is indicative of a neurogenic disease.

61. The method of claim 60, wherein the neurogenic disease is Alzheimer's disease.

62. The method of claim 50, wherein the template is further attached to a detectable label.

63. The method of claim 62, wherein the label is a fluorescent label.

64. The method of claim 62, wherein the label is a radioactive label.

65. The method of claim 50, wherein the sample is a biological sample.

66. The method of claim 50, wherein the sample comprises tissue taken from tissue extracts, urine, blood, phlegm, saliva, or cerebrospinal fluid.

67. The method of claim 50, wherein the method is performed at ambient temperature.

68. The method of claim 50, wherein the method is performed under thermo-cycling conditions.

69. A method of detecting Alzheimer's disease, comprising:
(a) contacting a sample with a modified detector molecule, such that a complex will be formed in the presence of a misfolded tau protein that is indicative of the disease, the modified detector molecule having two ends, a first end capable of binding to the misfolded tau protein, and a second detector end comprising a single-stranded DNA template, wherein the template is capable of being replicated by an RNA polymerase;
(b) contacting complex formed in (a) with an RNA polymerase such that the DNA template will be replicated in the presence of the misfolded tau protein; and
(c) detecting the replicated DNA template thereby indicating the presence of the misfolded tau protein and thus the presence of Alzheimer's disease.

70. A dipstick kit for detecting a target molecule comprising:
(a) an affinity ligand capable of detecting the target molecule attached to a dipstick; and
(b) a solution comprising a modified detector molecule capable of detecting the target molecule, attached to a liposome, wherein
(i) the modified detector molecule comprises two ends, a first end capable of binding the target molecule and a second end comprising a single-stranded DNA template, wherein the template is capable of being replicated by an RNA polymerase and
(ii) the liposome contains an RNA polymerase.

71. The dipstick kit of claim 70, wherein the target molecule is a protein.

72. The dipstick kit of claim 71, wherein the protein is indicative of a disease.

73. The dipstick kit of claim 71, wherein the protein is a tau protein.

74. The dipstick kit of claim 71, wherein the protein is a prion protein.

75. The dipstick kit of claim 70, wherein the target molecule is in a biological sample.

76. The dipstick kit of claim 75, wherein the biological sample is from tissue extracts, urine, blood, phlegm, saliva, or cerebrospinal fluid.

77. The dipstick kit of claim 70, wherein the kit is capable of being used at ambient temperature.

78. The dipstick kit of claim 70, wherein the kit is capable of being used under thermo-cycling conditions.

79. A dipstick kit for detecting a target molecule comprising:
(a) a modified detector molecule attached to a dipstick, wherein
(i) the modified detector molecule comprises two ends, a first end capable of binding the target molecule and a second end comprising a single-stranded DNA template, wherein the template is capable of being replicated by an RNA polymerase; and
(b) a solution comprising an affinity ligand capable of detecting the target molecule, attached to a liposome, wherein
(i) the liposome contains an RNA polymerase.

80. The dipstick kit of claim 79, wherein the target molecule is a protein.

81. The dipstick kit of claim 80, wherein the protein is indicative of a disease.

82. The dipstick kit of claim 80, wherein the protein is a tau protein.

83. The dipstick kit of claim 80, wherein the protein is a prion protein.

84. The dipstick kit of claim 79, wherein the target molecule is in a biological sample.

85. The dipstick kit of claim 84, wherein the biological sample is from tissue extracts, urine, blood, phlegm, saliva, or cerebrospinal fluid.

86. The dipstick kit of claim 79, wherein the kit is capable of being used at ambient temperature.

87. The dipstick kit of claim 79, wherein the kit is capable of being used under thermo-cycling conditions.


SEQUENCE LISTING (9 sequences)

SEQ ID NO: 1 - DNA, 221 bp, Artificial
  ARTIFICAL SEQUENCE VARIANT FROM A VIRUS
ggggaccccccggaaggggggacgaggtgcgggcacctcgtacgggagttcgaccgtgacgagtcacgggctagcgctttcgcgctctcccaggtgacgcctcgtgaagaggcgcgaccttcgtgcgtttcggcgacgcacgagaaccgccacgctgcttcgcagcgtggccccttcgcgcagcccgctgcgcgaggtgacccccgaaggggggttcccca

SEQ ID NO: 2 - DNA, 86 bp, Artificial
  ARTIFICAL SEQUENCE VARIANT FROM A VIRUS
gggttcatagcctattcggcttttaaaggacctttttccctcgcgtagctagctacgcgaggtgaccccccgaaggggggtgcccc

SEQ ID NO: 3 - DNA, 130 bp, Artificial
  ARTIFICAL SEQUENCE VARIANT FROM A VIRUS
gggttcatagcctattcggcttcgcgcatgggaatttgagggacgatggggaagtgggagcgcgttttaaaggacctttttccctcgcgtagctagctacgcgaggtgaccccccgaaggggggtgcccc

SEQ ID NO: 4 - DNA, 118 bp, Artificial
  ARTIFICAL SEQUENCE VARIANT FROM A VIRUS
gggttcatagcctattcggcttcgcgcccgtttataatacttagtgagcgcgttttaaaggacctttttccctcgcgtagctagctacgcgaggtgaccccccgaaggggggtgcccc

SEQ ID NO: 5 - DNA, 118 bp, Artificial
  ARTIFICAL SEQUENCE VARIANT FROM A VIRUS
gggttcatagcctattcggcttcgcgcccctggggtttgcctcaggagcgcgttttaaaggacctttttcccttgcgtagctagctacgcgaggtgaccccccgaaggggggtgcccc

SEQ ID NO: 6 - RNA, 196 nt, Artificial
  ARTIFICAL SEQUENCE VARIANT FROM A VIRUS
gggguuuccaaccggaauuugagggaugccuaggcaucccccgugcgucccuuuacgagggauugucgacucuagucgacgucugggcgaaaaauguacgagaggaccuuuucgguacagacgguaccugagggaugccuaggcaucccccgcgccgguuucggaccuccagugcguguuaccgcacugucgaccc

SEQ ID NO: 7 - DNA, 196 bp, Artificial
  ARTIFICAL SEQUENCE VARIANT FROM A VIRUS
ggggtttccaaccggaatttgagggatgcctaggcatcccccgtgcgtccctttacgagggattgtcgactctagtcgacgtctgggcgaaaaatgtacgagaggaccttttcggtacagacggtacctgagggatgcctaggcatcccccgcgccggtttcggacctccagtgcgtgttaccgcactgtcgaccc

SEQ ID NO: 8 - DNA, 86 bp, Artificial; identical to SEQ ID NO: 2
  ARTIFICAL SEQUENCE VARIANT FROM A VIRUS

SEQ ID NO: 9 - DNA, 85 bp, Artificial
  ARTIFICAL SEQUENCE VARIANT FROM A VIRUS
ggggcaccccccttcggggggtcacctcgcgtagctagctacgcgagggaaaaggtcctttaaaagccgcctaggctatgaaccc